(19) United States
(12) Patent Application Publication
(10) Pub. No.: US 2024/0393515 A1
(43) Pub. Date: Nov. 28, 2024

(54) OPTICAL FILTER STACK AND APPARATUS CONTAINING SAME

(71) Applicant: The OSLUV Project, St. Paul, MN (US)

(72) Inventors: Chia Hua M Pang, St. Paul, MN (US); Louis Goessling, Minneapolis, MN (US); Samuel Pellicori, Santa Barbara, CA (US); Aaron Collins, Minneapolis, MN (US)

(21) Appl. No.: 18/673,103

(22) Filed: May 23, 2024

Related U.S. Application Data
(60) Provisional application No. 63/504,419, filed on May 25, 2023.

(51) Int. Cl. G02B 5/28; A61L 9/20

(52) U.S. Cl. CPC G02B 5/28; A61L 9/20; A61L 2209/12

(57) ABSTRACT

An optical filter stack having layers of a high index dielectric material and layers of a low index dielectric material each having an optical thickness configured to together provide a transmittance curve having a transmittance of at least 70% at 222 nm and substantial transmittance of at least one wavelength longer than 240 nm, as measured at an incident angle of zero degrees. The disclosure also provides a reflective optical filter stack, a transmittance curve, a reflectance curve, a apparatus, and a method useful for germicidal far-UV applications, including inactivation of pathogens in air.

### CROSS-REFERENCE TO RELATED APPLICATIONS

This application claims the benefit of priority to U.S. Provisional Patent Application Ser. No. 63/504,419, filed May 25, 2023, the disclosure of which is incorporated herein in its entirety by reference.

### TECHNICAL FIELD

This document pertains generally, but not by way of limitation, to optical filter stacks, optical filters, and apparatus emitting filtered UV light. More specifically, but not by way of limitation, the present application relates to a filter useful for filtering far-UV light, including 222 nm light.

### BACKGROUND

Far-UV light has the potential to offer germicidal properties without causing harm to human skin or eyes. Considerable interest has grown around use of 222 nm light due to its ability to inactivate pathogens without deeply penetrating human tissue. On the other hand, light having wavelengths 230 nm and longer, particularly 240 nm or longer light, have increased penetration and can cause harm to human tissues. UV disinfecting devices configured for use in occupied spaces have been described that utilize optical filters that eliminate harmful radiation, particularly wavelengths at 230 nm and longer.

There is a need for improved optical filters for use with far-UV light.

### SUMMARY

The disclosure provides an optical filter stack having layers of a high refractive index dielectric material and layers a low refractive index dielectric material. The layers of a high refractive index dielectric material and layers a low refractive index dielectric material can be positioned so as to provide differences between refractive indexes of adjacent layers; for example, the layers can be alternating between high refractive index dielectric material and layers a low refractive index dielectric material. The optical thickness of the various layers is configured according to a transmittance curve that is useful for filtering UV radiation to inactivate pathogens while being safe for humans. The transmittance curve transmits antimicrobial UV radiation at one or more wavelength between 210 nm and 230 nm, particularly 222 nm, but also substantially transmits at least one wavelength longer than 240 nm, particularly one or more wavelength from 240 nm to 280 nm, as measured at an incident angle of zero degrees.

For example, the disclosure provides an optical filter stack having a plurality of layers of a high refractive index dielectric material and layers a low refractive index dielectric material, wherein the order of the layers and their optical thickness are configured to together provide a transmittance curve having:
(a) a transmittance of at least 70% at 222 nm and substantial transmittance of at least one wavelength longer than 240 nm, as measured at an incident angle of zero degrees;
(b) a transmittance of at least 70% at 222 nm, one or more wavelength from 230 nm to 245 nm having a transmittance about the same or greater than the transmittance at 222 nm, and each wavelength from 255 nm to 265 nm has a transmittance of less than 15% of the transmittance at 222 nm, as measured at an incident angle of zero degrees;
(c) a transmittance of at least 70% at 222 nm, a transmittance at one or more wavelengths from 240 nm to 245 nm that is about 85% or more of a peak transmittance from 222 nm to 245 nm, and a transmittance at one or more wavelengths from 255 nm to 265 nm that is about 15% or less of the peak transmittance from 222 nm to 245 nm, as measured at an incident angle of zero degree;
(d) a transmittance of at least 70% at 222 nm, a transmittance at one or more wavelength from 242 nm to 252 that is 50% of a peak transmittance from 222 nm to 242 nm, and a transmittance at one or more wavelengths from 255 nm to 265 nm that is at least 3% of the peak transmittance from 222 nm to 245 nm, as measured at an incident angle of zero degrees;
(c) a transmittance of at least 70% at 222 nm, a transmittance at one or more wavelengths from 255 nm to 265 nm that is 3% to 15% of a peak transmittance from 222 nm to 245 nm, and a transmittance at one or more wavelengths from 270 nm to 280 nm that is 20% or greater than the peak transmittance from 222 nm to 245 nm, as measured at an incident angle of zero degrees;
(f) a transmittance of at least 70% at 222 nm, a transmittance at one or more wavelengths from 255 nm to 265 nm that is 3% to 15% of a peak transmittance from 222 nm to 245 nm, and a transmittance at one or more wavelengths from 270 nm to 370 that is 90% or greater than the peak transmittance from 222 nm to 245 nm, as measured at an incident angle of zero degrees;
(g) a transmittance of at least 70% at 222 nm, a transmittance at one or more wavelength from 230 nm to 240 that is 50% of a peak transmittance from 222 nm to 230 nm, a transmittance at one or more wavelengths from 245 nm to 260 nm that is 3% to 15% of the peak transmittance from 222 nm to 230 nm, a transmittance at one or more wavelengths from 270 nm to 280 nm that is 20% or greater than the peak transmittance from 222 nm to 230 nm, and a transmittance at one or more wavelengths from 270 nm to 370 nm that is 90% or greater than the peak transmittance from 222 nm to 230 nm, as measured at an incident angle of zero degrees; or
(h) any combination thereof.

The disclosure further provides an apparatus for inactivating a pathogen in air, comprising a far-UV radiation source that emits radiation through a window comprising an optical filter stack having plurality of layers of a high refractive index dielectric material and layers a low refractive index dielectric material each having an optical thickness configured to together provide a peak emission at about 222 nm, and an emission at one or more wavelengths from 250 nm to 265 nm having an intensity of 0.1% or more of the emission at 222 nm, as measured upon treatment with an unfiltered KrCl excimer emission, as measured at an incident angle of zero degrees.

The disclosure also provides a method for inactivating a pathogen in air, comprising irradiating air in a human-use space with UV radiation having a peak emission at 222 nm, an emission at one or more wavelengths from 237 nm to 245 nm having an intensity of 1% or more of the emission at 222 nm, and an emission at one or more wavelengths from 250 nm to 265 nm having an intensity of 0.1% or more of the emission at 222 nm.

Various aspects of the presently described optical filter, apparatus, and method can have one or more of the advantages of (i) utilizing a simple, economical filter stack having a reduced number of layers, (ii) improved total transmittance of germicidal light, (iii) improved transmittance of 222 nm light, (iv) improved tolerance of wider-angle incident light. Use of such filter in a far-UV light apparatus can have the further advantage of providing a way to safely irradiate pathogens in occupied spaces using far-UV light without posing a danger to human eyes or skin, and can do so while harnessing longer wavelength radiation.

Additionally, in various examples, the optical filter, apparatus, or method can be configured to have a reflectance curve that reflects visible light to achieve one or more of the advantages of (i) solving the problem of purple-looking far-UV light sources; (ii) provide a far-UV light source that appears more like a conventional light that illuminates in the visible light range; and (iii) utilize room light via reflecting it back to the room and so avoid an apparatus having a bulb or light source that generates visible light, or avoid an apparatus that transmit its own visible light through its optical components.

### BRIEF DESCRIPTION OF THE FIGURES

FIG. 1 provides a diagram of an example optical filter stack.

FIG. 2 provides a diagram of an optical filter stack deposited on a substrate.

FIG. 3 provides a schematic of a far-UV lamp having an optical filter.

FIG. 4 provides a transmittance curve (FIG. 4A) for a first example optical filter stack and a normalized emission curve (FIG. 4B) for a KrCl far-UV lamp utilizing the first example optical filter stack.

FIG. 5 provides a transmittance curve (FIG. 5A) for a second example optical filter stack and a normalized emission curve (FIG. 5B) for a KrCl far-UV lamp utilizing the second example optical filter stack.

FIG. 6 provides a transmittance curve (FIG. 6A) for a third example optical filter stack and a normalized emission curve (FIG. 6B) for a KrCl far-UV lamp utilizing the third example optical filter stack.

FIG. 7 provides a transmittance curve (FIG. 7A) for a fourth example optical filter stack and a normalized emission curve (FIG. 7B) for a KrCl far-UV lamp utilizing the fourth example optical filter stack.

FIG. 8 provides a transmittance curve (FIG. 8A) for a fifth example optical filter stack and a normalized emission curve (FIG. 8B) for a KrCl far-UV lamp utilizing the fifth example optical filter stack.

FIG. 9 provides a transmittance curve (FIG. 9A) for a sixth example optical filter stack and a normalized emission curve (FIG. 9B) for a KrCl far-UV lamp utilizing the sixth example optical filter stack.

FIG. 10 provides transmittance curves for a first set of manufactured example optical filter stacks.

FIG. 11 provides transmittance curves for a second set of manufactured example optical filter stacks.

FIG. 12 provides an example optical filter stack deposited on two sides of a substrate.

### DETAILED DESCRIPTION OF THE INVENTION

The present disclosure provides an optical filter stack useful for filtering far-UV radiation. An optical filter stack is an optical device designed to selectively transmit or reflect light based on wavelength. The device has a number of layers of high and low refractive index materials arranged so as to provide a difference in refractive index between adjacent layers. The optical thickness of each layer can be tuned to control the wavelengths that are transmitted or reflected. Optical thickness corresponds to the refractive index multiplied by its physical thickness. The design of layers in the filter stack can be based on a quarter wave layer design or a non-quarter wave layer design. Indeed, in various aspects described herein, the filter stack can be a non-quarter wave filter stack. As incident light passes through the filter stack, it is partially reflected and partially transmitted at the interface between alternating layers. Those skilled in the art will understand that by controlling the thickness of each iterative layer, the filter stack can be designed to a desired transmittance curve. See, for example, H. Angus Macleod, Thin-Film Optical Filters, 3rd ed. 2001. Taylor and Francis Group, New York, NY, which is incorporated by reference herewith in its entirety. The layers are not necessarily strictly alternating; suitable results can be achieved with some deviation from a strictly alternating pattern of high and low refractive index layers, but for general purposes the performance of the optical filter stack relies on differences between higher and lower refractive index materials, so it is the alternating relationship that imparts control of the transmittance curve. In various aspects, multiple types of high or low refractive index materials may be used or additional layers may be used, such as layers with moderate refractive index, transparent layers, or reflective layers. The additional layers may be used in between or in place of the higher or lower refractive index materials, provided that the layers are configured with appropriate thickness and ordering so as to provide the target transmittance curve and/or reflectance curve.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

As used herein, the term “visible light” is defined as light having a wavelength of from 380 nm to 750 nm. In some alternative examples, visible light can have a wavelength greater than 380 nm and up to 700 nm, or greater than 400 nm and up to 700 nm. In various examples, the far-UV radiation source, apparatus, filter stack, and the like, relate to electromagnetic radiation that is outside of the visible light range. In various examples, the far-UV radiation source produces far-UV radiation but not visible light, and the filter can be configured to pass or stop various wavelengths of far-UV radiation, but need not necessarily pass or stop visible light. Yet, in various other aspects, the far-UV radiation source, apparatus, filter stack, and the like, can further involve a visible light source and visible light emission.

As used herein, the term “ultraviolet radiation” or “UV radiation” refers to electromagnetic radiation having one or more wavelength of about 100 nm to about 400 nm. UV radiation can further be subdivided into several categories based on wavelength: UVA (about 315 nm to about 400 nm), UVB (about 280 nm to about 315 nm), UVC (about 200 nm to about 280 nm), far-UV (about 200 nm to about 230 nm), vacuum UV (about 100 nm to about 200 nm), extreme UV (about 10 nm to about 100 nm). “Germicidal UV” can refer to about 200 nm to about 400 nm. In various aspects and contexts, UV radiation can refer to various sub-ranges of UV radiation, for example, 180 nm to 400 nm, 200 nm to 400 nm, 210 nm to 400 nm, 200 nm to 370 nm, 200 nm to 300 nm, or 210 nm to 280 nm. Radiation sources suitable for the presently described apparatus, system and method would not typically emit vacuum UV or extreme UV. As such, in various aspects the UV radiation source can be a UV radiation source that does not emit one or more type of UV light, such as a radiation source that does not emit vacuum UV or extreme UV. For example, the UV radiation source can emit radiation comprising or consisting of one or more wavelengths between 200 nm and 400 nm. UV radiation sources that do not emit vacuum UV. A given radiation source can emit a spectrum having wavelengths according to multiple categories of light. For example, a far-UV radiation source may emit radiation having wavelengths corresponding to UVA, UVB, or UVC.

As used herein, the “optical thickness” or “OT” of a layer of an optical filter stack corresponds as follows: OT=refractive index at the target wavelength multiplied by the physical thickness of the given layer. In various examples herein, 260 nm at an incident angle of 0 is used as a reference wavelength.

As used herein, “transmittance” refers to a percentage of light passing through an optical material, which can be an optical filter. Transmittance is a ratio of the intensity of transmitted light over the intensity of the incident light at a given wavelength, or range of wavelengths, for a given incident angle or angles. Transmittance is a ratio and thus unitless. It can be reported as a percentage or a fraction. Transmittance can be measured via spectroscopy or it can be simulated from first principles given known optical properties of a given material. In various examples, transmittance can be determined from power (W/W) or irradiance ((W/m²)/(W/m²)). Computational and mathematical tools for determining transmittance are known to those skilled in the art. Unless otherwise specified, transmittance values described herein can be determined from a power ratio. Transmittance can also be described in relation to a peak transmittance. Depending on context, transmittance can refer to one or more wavelengths, one or more spectral region, or can where specified refer to radiant transmittance. A transmittance curve describes transmittance across a range of wavelengths, or transmittance across a spectrum.

As used herein, “reflectance” refers to a ratio of the amount of light reflected out of the total amount of incident light. Reflectance can be measured via spectroscopy or it can be simulated from first principles given known optical properties of a given material. Computational and mathematical tools for determining reflectance are known to those skilled in the art.

As used herein, “emission” in the context of light emitted through an optical material refers to an amount of light emitted through the optical filter based on a particular source spectrum of light incident to the optical material. Emission can be quantified in terms of irradiance or optical power flux, but in this document is typically compared to another emission to provide an emission curve. For example, emissions can be described as a percentage of the amount of light emitted at one wavelength relative to the amount emitted at another; for example: I₂₅₈ nm/I₂₂₂ nm, which results in a unitless value.

As used herein, “irradiance” is a measurement of power delivered to a surface per unit area, typically in units of ‘W/m²’ (watts per square meter), mW/m² or μW/cm². The surface can be an actual surface, e.g., of an object, or a given imaginary surface. Irradiance is one approach to describing the intensity of light. As a surface with a fixed area moves away from a light source the power delivered to it decreases as the square of the distance. Irradiance values depend on the orientation of the surface relative to the light source, declining with the difference between the surface's orientation and the orientation that would place it normal to incident rays from the light source. In some contexts, when irradiance is described based on a given distance from the radiation source, the surface can be understood to be an imaginary planar area at a distance from a radiation source and oriented facing the light source so as to be normal to incident rays from the source. In other contexts, when irradiance is described with respect to an object, the relevant surface can be understood to correspond to the surface orientation of such object, which can include walls, floors, ceilings, objects, or persons. Irradiance can be arrived at from a radiance value by specifying a distance and orientation for the irradiance value. Irradiance can also be arrived at from total optical power with knowledge of the distance from the source, orientation of the surface, and the relative position of the surface in the radiation pattern of the source. Under a fixed spectra or wavelength this can be equated with a certain photon arrival rate for a certain surface in photons/(second·cm²). When characterizing optical filters or describing how performance differs across wavelengths, use of “spectral irradiance” is typically appropriate. In various contexts, reference to irradiance at particular wavelengths can be understood to refer to “spectral irradiance”. Spectral irradiance refers to irradiance at a given wavelength or wavelengths. Spectral irradiance can be useful in order to reflect that effects or properties differ at different wavelengths. While irradiance describes power per area (uW/cm\hat{ }2), spectral irradiance can be described as power per area in nanometer bin widths of the spectrum (uW/cm\hat{ }2·nm). Irradiance and spectral irradiance are related because integrating across a certain spectral irradiance wavelength range, e.g., 180-400 nm, will provide irradiance. Irradiance can also be specified as being in a ‘radiant’ context integrated over the entire wavelength range. As used herein, “total irradiance” refers to irradiance across the 180-400 nm wavelength range, which can typically be sufficient to describe the entire relevant wavelength range of light from far-UV sources; it can be reported in units of W/cm2 at a given distance from the light source. It can also be useful to refer to irradiance at specific wavelengths or of a specific narrow spectral series, such as a 222 nm or narrow band 222 nm light. In various aspects, irradiance, various spectral irradiance, total irradiance, and the like can be useful for determining an instantaneous amount of radiation provided to a surface or for determining an average amount of radiation provided to a surface over a given period of time. Irradiance, spectral irradiance, total irradiance, and the like can also be used to measure or determine the disinfectant efficacy of a far-UV lamp, which can contain an optical filter, as well as its contribution to effective air changes per hour (eACH) in a given space, where such determinations can optionally be weighted based on the spectral profile of the light. In various aspects, irradiance, spectral irradiance, total irradiance, and the like can be useful for controlling disinfectant power of the far-UV lamp, such as via maintaining operation within a given germicidal efficacy profile and maintaining operation within a given safety profile.

As used herein, “fluence rate” is a measurement of power delivered to the surface area of a sphere, typically in units of “W/m²” (watts per square meter), mW/m² or μW/cm². This metric can be similar to “irradiance”, can share the same unit, and can be similarly used. However, whereas irradiance measures the rate of energy delivery to an oriented surface, fluence rate measures the rate of energy delivery to the surface of a sphere, and is therefore not dependent on the orientation of the measurement in space. When an irradiance measurement is taken for a surface pointing directly at a radiation source (i.e. such that the surface is normal to incident rays) and in the absence of reflections, the irradiance measurement and fluence rate measurements for that point will match. Another way to think of this is that irradiance only measures photons passing through a surface from one direction, and fluence rate measures photons passing through the surface of a spherical volume from any direction. Fluence rate as used herein is used in a ‘radiant’ context integrated over a relevant UV spectral range, e.g., often conveniently integrated across 180 nm to 400 nm. In various aspects, fluence rate is useful for determining an instantaneous amount of radiation provided to a volume or for determining an average amount of radiation provided to a volume over a given period of time. In various aspects, fluence rate is useful for determining instantaneous viral inactivation power of a UV light source, and correlates to eACH for the measured volume.

As used herein, “total optical power”, “total optical flux” or “total optical power flux” refers to the photon generation rate of a UV radiation source across the 180-400 nm wavelength range in units of mW. It can be used to describe the disinfectant power of a lamp. This can be used as a measurement of the total power output of a UV radiation source or other device. This is distinct from the ‘wall power’ consumption of the lamp which is not related to this term in any meaningful way besides bounding it above.

As used herein, “effective irradiance” refers to the contribution of a given device to exposure limits, which can dictate the number of lamps suitable for deployment in a given space or dictate the allowable exposure time which a light may be used in an occupied space. As described herein, effective irradiance, “E_{ff}”, or “Eₛ”, can be calculated by measuring pointwise irradiance between 180-400 nm weighted by a factor corresponding to potential damage to the eyes or skin.

The filter stack, optical filter, far-UV apparatus, and method described herein can be useful for inactivation of airborne pathogens in an occupiable space. As used herein, the term “pathogen” includes viruses, bacteria, infectious microbes, fungi, protozoa, parasites, and other agents, or components thereof, that are capable of causing disease or infection in humans or animals. Pathogens includes pathogens in droplets, pathogens in aerosols, and free pathogens in air. As used herein, the term “inactivation” refers to a partial or complete reduction in a pathogen's ability to cause disease or infection. Without intending to limit to any particular theory of mechanism, inactivation may be achieved by damaging or destroying the pathogen's genetic material, membrane, or other structures through exposure to far-UV radiation. In some contexts, inactivation can refer to inactivating a pathogen's ability to reproduce, replicate, enter a cell, or evade the immune system. Inactivation of pathogens can be useful for disinfection or decontamination purposes. As used herein, “human-use space” or “occupiable space” is an area that is designed or intended for use by humans. Examples include, but are not limited to, rooms, restaurants, schools, workplaces, offices, hospitals, cafeterias, event space, bars, theaters, gyms, retail space, public transportation vehicles, public transportation stations, airports, and airplanes. Human-use space or occupiable space can include indoor space and outdoor space. Human-use space or occupiable space can be empty, or it can be occupied space, for example by one or more occupants. Indoor space can include indoor space that is open, ventilated, or non-ventilated. In various examples, indoor space can have windows, or mechanical ventilation such as a HVAC system including air supplies and air returns. An occupiable space or occupied space can also refer to an animal-use space or plant-use space. The term “treatment space” refers to the occupiable space having the air that is irradiated to inactivate pathogens in air. As such, the volume of the treatment space refers to the volume of the entire human-use space, e.g., a room, whereas the volume of irradiated air refers only to the portion of the treatment space that is subject to irradiation. Occupiable space can have certain areas expected to be occupied. In various aspects, areas of an occupiable space that may be expected to be occupied can include seats, desks, tables, bar tops, waiting areas, patient beds, reception space, checkout areas, cubicles, stairways, walkways, and elevators. Typically, areas of expected human occupancy are less than 7 feet from a floor.

Reference will now be made in detail to certain embodiments of the disclosed subject matter, several examples of which are illustrated in part in the accompanying drawings. While the disclosed subject matter will be described in conjunction with the enumerated claims, it will be understood that the exemplified subject matter is not intended to limit the claims to the disclosed subject matter. Moreover, those of skill in the art will understand that various modifications and variations are within the scope of the present invention, which need not be limited to the specific examples described herein.

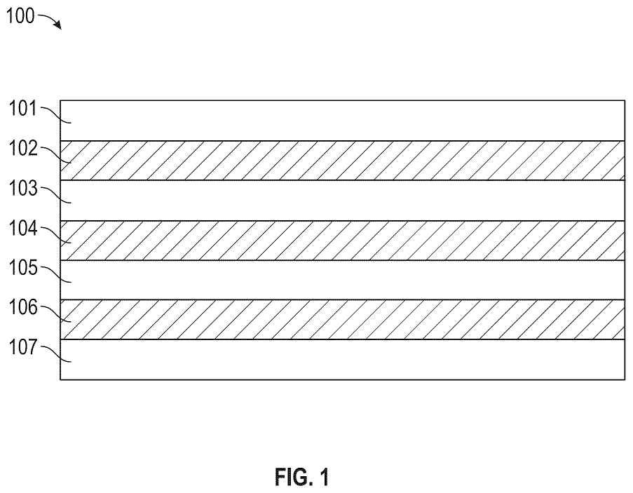

FIG. 1 illustrates an example of an optical filter stack 100. The filter stack is configured having layers that alternate between a high index dielectric material and a low index dielectric material. The filter stack can have 5 to 50 such alternating layers. The filter stack has n number of layers, each having a thickness X₁, X₂, X₃, X₄ . . . Xₙ. The filter stack is configured to receive incident light at the first layer 101 and transmit at least a portion of it through the remaining layers. The non-limiting example illustrated in FIG. 1 has seven layers, but other numbers of layers are possible. The first layer 101 is a dielectric material having a low refractive index and thickness X₁, and the second layer 102 is a dielectric material having a high refractive index and thickness X₂. The figure illustrates additional layers, which are numbered 103, 104, 105, 106, and 107, each alternating between low and high index dielectric materials. In various aspects, the filter stack can contain yet additional layers beyond those expressly shown in present figure; such further layers may be referenced by numbers 108, 109, 110, 111, 112, 113, 114, 115, 116, 117, 119, . . . , etc. The layers each have a thickness such that when taken together can provide a transmittance curve according to one, or any combination, of the following:
(a) a transmittance of at least 70% at 222 nm and substantial transmittance of at least one wavelength longer than 240 nm, as measured at an incident angle of zero degrees;
(b) a transmittance of at least 70% at 222 nm, one or more wavelength from 230 nm to 245 nm having a transmittance about the same or greater than the transmittance at 222 nm, and each wavelength from 255 nm to 265 nm has a transmittance of less than 15% of the transmittance at 222 nm, as measured at an incident angle of zero degrees;
(c) a transmittance of at least 70% at 222 nm, a transmittance at one or more wavelengths from 240 nm to 245 nm that is about 85% or more of a peak transmittance from 222 nm to 245 nm, and a transmittance at one or more wavelengths from 255 nm to 265 nm that is about 15% or less of the peak transmittance from 222 nm to 245 nm, as measured at an incident angle of zero degree;
(d) a transmittance of at least 70% at 222 nm, a transmittance at one or more wavelength from 242 nm to 252 that is 50% of a peak transmittance from 222 nm to 242 nm, and a transmittance at one or more wavelengths from 255 nm to 265 nm that is at least 3% of the peak transmittance from 222 nm to 245 nm, as measured at an incident angle of zero degrees;
(e) a transmittance of at least 70% at 222 nm, a transmittance at one or more wavelengths from 255 nm to 265 nm that is 3% to 15% of a peak transmittance from 222 nm to 245 nm, and a transmittance at one or more wavelengths from 270 nm to 280 nm that is 20% or greater than the peak transmittance from 222 nm to 245 nm, as measured at an incident angle of zero degrees;
(f) a transmittance of at least 70% at 222 nm, a transmittance at one or more wavelengths from 255 nm to 265 nm that is 3% to 15% of a peak transmittance from 222 nm to 245 nm, and a transmittance at one or more wavelengths from 270 nm to 370 that is 90% or greater than the peak transmittance from 222 nm to 245 nm, as measured at an incident angle of zero degrees;
(g) a transmittance of at least 70% at 222 nm, a transmittance at one or more wavelength from 230 nm to 240 that is 50% of a peak transmittance from 222 nm to 230 nm, a transmittance at one or more wavelengths from 245 nm to 260 nm that is 3% to 15% of the peak transmittance from 222 nm to 230 nm, a transmittance at one or more wavelengths from 270 nm to 280 nm that is 20% or greater than the peak transmittance from 222 nm to 230 nm, and a transmittance at one or more wavelengths from 270 nm to 370 nm that is 90% or greater than the peak transmittance from 222 nm to 230 nm, as measured at an incident angle of zero degrees; or (h) any combination thereof.

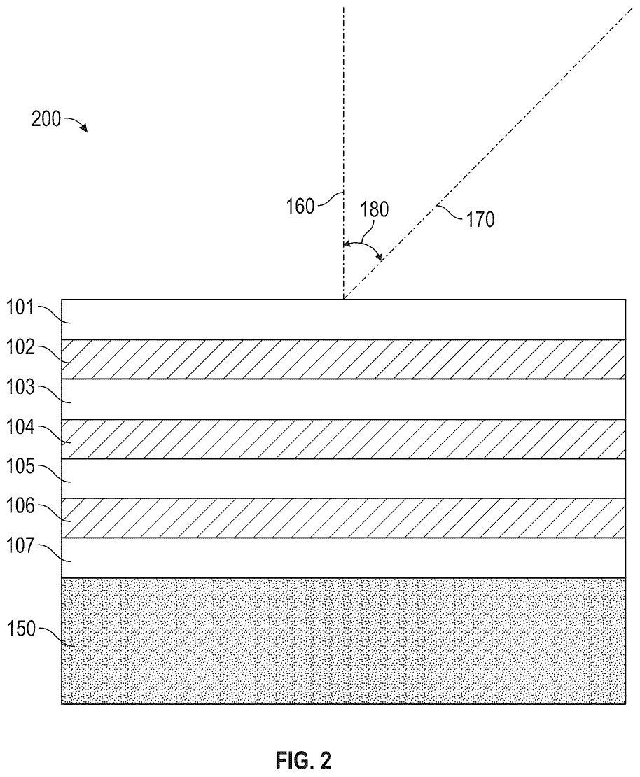

FIG. 2 illustrates an example optical filter 200, which comprises an optical filter stack 100 disposed on a substrate 150. The filter stack can have 5 to 50 layers that alternate between a high index dielectric material and a low index dielectric material. FIG. 2 shows, as a non-limiting example, a first layer 101 as an outermost layer and the last layer 107 as an innermost layer disposed upon the substrate 150. Alternatively, the first layer 101 can be disposed upon the substrate 150 and the outermost layer can be the last layer 107. Although the example in FIG. 2 illustrates seven layers, other numbers of layers are possible. Light can encounter the optical filter stack 100 as on-axis incident light 160 or off-axis incident light 170, which has an angle of incidence 180, or any combination thereof. While this example illustrates both on-axis incident light 160 and off-axis light 170, this distinction is primarily for illustration only, in practice light provided to an optical filter can represent a combination of incident approaches and optical filters can be understood to be generally tolerant and operable across a reasonable variety of light approaches The substrate 150 is a transparent material. The transparent material can be crystalline material such as quartz, or a glass such as fused silica. The substrate can serve as a mechanical support for manufacturing or maintaining the structure of the optical filter stack, and it can serve as a means for positioning the optical filter stack upon a far-UV generating device.

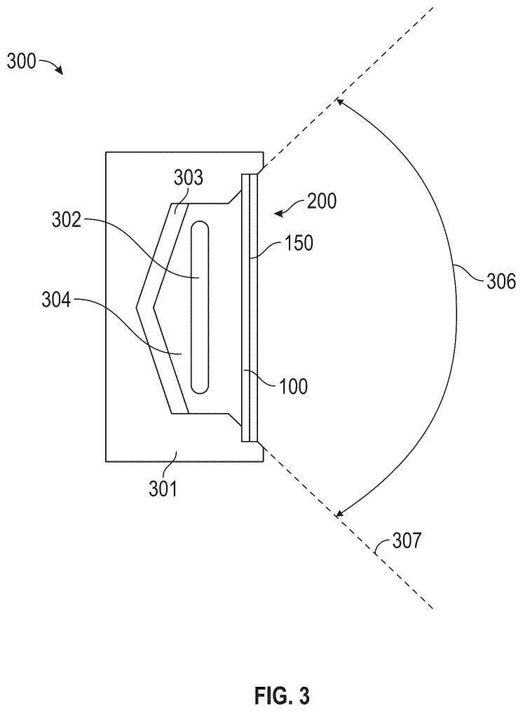

FIG. 3 illustrates an example far-UV light apparatus 300, which can be useful for inactivating pathogens. The apparatus has a housing 301 that contains a far-UV radiation source 302 and, optionally, a reflector 303. The far-UV radiation source 302 can be an excimer lamp, such as a barrier discharge lamp. The far-UV radiation source 302 can be a KrCl excimer lamp or a KrBr excimer lamp. The housing 301 defines an open chamber 304, which is configured to accommodate the far-UV radiation source 302 and provide an aperture through which emitted radiation is directed through an optical filter 200. The optical filter 200 is secured to the housing and covers the aperture. The open chamber 304 can be equipped with a reflector 303 to assist in directing and maximizing emitted radiation. The apparatus 300 emits radiation in a broadcast pattern defined by an angle 306 between edges 307 of the broadcast pattern with respect to a given wavelength of light, such as 222 nm. The resulting broadcast pattern is defined by the geometry of the apparatus 300, its permitted angles of light incident upon the optical filter 200, the transmittance through the filter at the given wavelength, and the resulting emission. In various aspects, the far-UV light apparatus provides a broadcast angle of greater than 45 degrees at a transmittance of greater than 80% at 222 nm. In various configurations, the far-UV light apparatus 300 comprises a filter 200 that provides a transmittance of greater than 80% at 222 nm for light having an incident angle of 45 degrees.

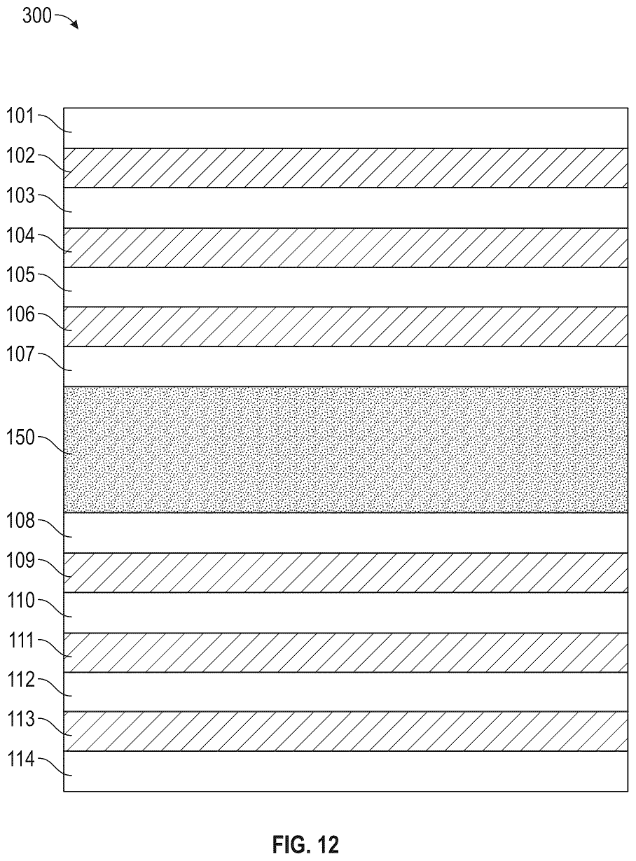

FIG. 12 illustrates an example optical filter 300, which comprises an optical filter stack 100 disposed on two sides of a substrate 150. The filter stack can have 5 to 50 layers, per side, or total across both sides, that alternate between a high index dielectric material and a low index dielectric material. FIG. 12 shows, as a non-limiting example, a first layer 101 as an outermost layer on a first side of the substrate and the last layer 107 as an innermost layer on the first side of the substrate 150. FIG. 12 further shows, as a non-limiting example, a first layer 108 as an innermost layer on a second side of the substrate and the last layer 114 as an outermost layer on the second side of the substrate 150. The layers on each side of the substrate, together provide an optical filter stack that provides a desired optical filter transmittance curve or other behavior. Various alternative configurations are possible, many of which can be configured to provide equivalent results. For example, the layers on each side can be moved around in various manners, for example, the innermost layer of one side can be configured so that it is an outermost layer of the other side, and the outermost layer of one can be configured so that it is the first layer 101 can be disposed upon the substrate 150 and the outermost layer can be the last layer 107. Although the example in FIG. 12 illustrates seven layers on each side, other numbers of layers are possible. Light can encounter the optical filter stack 100 as on-axis incident light 160 or off-axis incident light 170, which has an angle of incidence 180, or any combination thereof. As noted above, unless otherwise specified, optical filters can be tolerant of a range of incident light approaches. The substrate 150 is a transparent material. The transparent material can be crystalline material such as quartz, or a glass such as fused silica. The substrate can serve as a mechanical support for manufacturing or maintaining the structure of the optical filter stack, and it can serve as a means for positioning the optical filter stack upon a far-UV generating device.

The filter stack can be configured having layers that alternate between a high index dielectric material and a low index dielectric material. It can have 5 to 50 such alternating layers disposed one or more sides of disposed on a substrate; for example: it can have 5 to 50 layers comprising low index layers and high index layers on a single side of a substantially planar substrate, or it can have 5 to 50 layers comprising low index layers and high index layers distributed across two sides of a substantially planar substrate. The combination of layer separately disposed can be understand as representing a single filter stack, when taken together. The filter stack has n number of layers, each having a thickness X₁, X₂, X₃, X₄ . . . . Xₙ. The filter stack is configured to receive incident light at the first layer 101 and transmit at least a portion of it through the remaining layers on the first side, then through the substrate 150, then through the layer 108, then through the remaining layers of the second side. The non-limiting example illustrated in FIG. 12 has seven layers per side, but other numbers of layers are possible. The first layer 101 is a dielectric material having a low refractive index and thickness X₁, and the second layer 102 is a dielectric material having a high refractive index and thickness X₂. The figure illustrates additional layers, which are numbered 103, 104, 105, 106, and 107, each alternating between low and high index dielectric materials. In various aspects, the filter stack can contain yet additional layers beyond those expressly shown in present figure; such further layers may be referenced by numbers 115, 116, 117, 119, . . . , etc. In various aspects, a dual-sided optical filter stack can have two sides (e.g., front and back of a planar substrate) that are configured to together provide a transmittance curve according to one, or any combination, of the following:
(a) a transmittance of at least 70% at 222 nm and substantial transmittance of at least one wavelength longer than 240 nm, as measured at an incident angle of zero degrees;
(b) a transmittance of at least 70% at 222 nm, one or more wavelength from 230 nm to 245 nm having a transmittance about the same or greater than the transmittance at 222 nm, and each wavelength from 255 nm to 265 nm has a transmittance of less than 15% of the transmittance at 222 nm, as measured at an incident angle of zero degrees;
(c) a transmittance of at least 70% at 222 nm, a transmittance at one or more wavelengths from 240 nm to 245 nm that is about 85% or more of a peak transmittance from 222 nm to 245 nm, and a transmittance at one or more wavelengths from 255 nm to 265 nm that is about 15% or less of the peak transmittance from 222 nm to 245 nm, as measured at an incident angle of zero degree;
(d) a transmittance of at least 70% at 222 nm, a transmittance at one or more wavelength from 242 nm to 252 that is 50% of a peak transmittance from 222 nm to 242 nm, and a transmittance at one or more wavelengths from 255 nm to 265 nm that is at least 3% of the peak transmittance from 222 nm to 245 nm, as measured at an incident angle of zero degrees;
(c) a transmittance of at least 70% at 222 nm, a transmittance at one or more wavelengths from 255 nm to 265 nm that is 3% to 15% of a peak transmittance from 222 nm to 245 nm, and a transmittance at one or more wavelengths from 270 nm to 280 nm that is 20% or greater than the peak transmittance from 222 nm to 245 nm, as measured at an incident angle of zero degrees;
(f) a transmittance of at least 70% at 222 nm, a transmittance at one or more wavelengths from 255 nm to 265 nm that is 3% to 15% of a peak transmittance from 222 nm to 245 nm, and a transmittance at one or more wavelengths from 270 nm to 370 that is 90% or greater than the peak transmittance from 222 nm to 245 nm, as measured at an incident angle of zero degrees;
(g) a transmittance of at least 70% at 222 nm, a transmittance at one or more wavelength from 230 nm to 240 that is 50% of a peak transmittance from 222 nm to 230 nm, a transmittance at one or more wavelengths from 245 nm to 260 nm that is 3% to 15% of the peak transmittance from 222 nm to 230 nm, a transmittance at one or more wavelengths from 270 nm to 280 nm that is 20% or greater than the peak transmittance from 222 nm to 230 nm, and a transmittance at one or more wavelengths from 270 nm to 370 nm that is 90% or greater than the peak transmittance from 222 nm to 230 nm, as measured at an incident angle of zero degrees; or
(h) transmittance of at least some 222 nm, and transmittance at one or more wavelength from 230 nm to 240 that is 50% of a peak transmittance from 222 nm to 230 nm, a transmittance at one or more wavelengths from 245 nm to 260 nm that is 3% to 15% of the peak transmittance from 222 nm to 230 nm, a transmittance at one or more wavelengths from 270 nm to 280 nm that is 20% or greater than the peak transmittance from 222 nm to 230 nm, and a transmittance at one or more wavelengths from 270 nm to 370 nm that is 90% or greater than the peak transmittance from 222 nm to 230 nm, as measured at an incident angle of zero degrees;
(i) transmittance of at least some 222 nm, a transmittance at one or more wavelength from 280 nm to 300 nm that is greater than the peak transmittance of 220 or the peak transmittance from 222 nm to 230 nm, a transmittance at one or more wavelengths from 245 nm to 260 nm that is 0.1% to 15% of relative to the peak transmittance from 222 nm to 230 nm;
(j) transmittance of at least some 222 nm, a transmittance at one or more wavelength from 300 nm to 360 nm that is greater than the peak transmittance of 220 or the peak transmittance from 222 nm to 245 nm, a transmittance at one or more wavelengths from 260 nm to 280 that is 0.1% to 15% of relative to the peak transmittance from 222 nm to 245 nm; or
(k) any combination thereof.

In various further aspects, the single-sided filter stack or dual-sided filter stack is configured to provide a reflectance curve according to one, or any combination, of the following:
(a) a reflectance of at least 50% for one or more wavelength of visible light having a wavelength greater than 450 nm;
(b) a reflectance of at least 50% at one or more wavelength from about 430 nm to about 750 nm;
(c) a reflectance curve having a reflectance of at least 60% at one or more wavelength from about 430 nm to about 750 nm;
(d) a reflectance curve having a reflectance of at least 70% at one or more wavelength from about 430 nm to about 750 nm;
(c) a reflectance curve having a reflectance of at least 60% for each wavelength from about 450 nm to about 580 nm, a reflectance curve having a reflectance of at least 70% for each wavelength from about 460 nm to about 570 nm;
(f) a reflectance curve having a reflectance of at least 80% for each wavelength from about 540 nm to about 560 nm; or
(g) any combination thereof.

### Optical Filter Stack

The filter stack is an optical component structured with thin solid film layers of a high refractive index material and a low refractive index material. It functions to transmit desired UV wavelengths while attenuating undesired UV wavelengths. Each layer is designed to selectively transmit or attenuate (e.g., by reflecting or absorbing) particular ranges of wavelengths so as to achieve a desired transmittance curve profile. The thickness of the various layers of the high refractive index dielectric material and the low refractive index dielectric material determines the resulting transmittance curve. A given transmittance curve can be predictably achieved by adjusting thickness of the layers to provide the desired filter response. See, for example, H. Angus Macleod, Thin-Film Optical Filters, 3rd ed. 2001. Taylor and Francis Group, New York, NY, which is incorporated by reference herewith in its entirety. The transmittance curve of a given filter stack can be reasonably predicted via modeling, including computer modeling. Such modeling can also identify suitable filter stack configurations based on a desired transmittance curve. Those skilled in the art will readily understand that the optical properties of each layer can be precisely tuned by adjusting the material composition of the layer, its thickness, and the order the layers are arranged. In various aspects, the filter stack is a non-quarter wave filter stack.

The filter stack can have from 5 to 50 layers. In various aspects, the filter stack can have at least or about 5, 6, 7,8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 layers. In further aspects, the filter stack can have less than 6, 7,8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 layers. For example, the filter stack can be 30 or fewer layers, 20 or fewer layers, 8 to 20 layers, 9 to 16 layers, or 9 to 12 layers. The filter stack layers, taken together, can have a total thickness of at least or about 100 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, or 1000 nm. In further aspects, the filter stack layers, taken together, can have a total thickness of 100 nm to 5000 nm. In further aspects, the filter stack layers, taken together, can have a total thickness less than 450 nm, 500 nm, 550 nm, 600 nm, 650 nm, 700 nm, 750 nm, 800 nm, 850 nm, 900 nm, 1000 nm, 1100 nm, 1200 nm, 1300 nm, 1400 nm, 1500 nm, 1600 nm, 1700 nm, 1800 nm, 1900 nm, or 2000 nm. Each layer has an optical thickness such that when all layers are taken together they provide the target transmittance profile.

The filter stack can provide a peak transmittance at one or more wavelength between 210 nm and 250 nm. In various aspects, the filter stack can be configured to provide a peak transmittance at about 210 nm, 220 nm, 221 nm, 222 nm, 223 nm, 224 nm, 225 nm, 226 nm, 227 nm, 228 nm, 229 nm, 230 nm, 231 nm, 232 nm, 233 nm, 234 nm, 235 nm, 236 nm, 237 nm, 238 nm, 239 nm, 240 nm, 241 nm, 242 nm, 243 nm, 244 nm, 245 nm, 246 nm, 247 nm, 248 nm, 249 nm, or 250 nm, when measured at 0, 5, 10, 15, 20, 25, 30, 35, or 40 degrees incident light, or between any values therein. The peak transmittance can be a local maximum or a global maximum. In various aspects, the local maximum is a maximum in the range of 200 nm to 280 nm. In various aspects, the local maximum is a maximum in the range of 200 nm to 250 nm. In further aspects, the filter stack can provide a transmittance greater than, or about, 65%, 70%, 75%, 80%, 85%, 90%, or 95%, at about 210 nm, 220 nm, 221 nm, 222 nm, 223 nm, 224 nm, 225 nm, 226 nm, 227 nm, 228 nm, 229 nm, 230 nm, 231 nm, 232 nm, 233 nm, 234 nm, 235 nm, 236 nm, 237 nm, 238 nm, 239 nm, 240 nm, 241 nm, 242 nm, 243 nm, 244 nm, 245 nm, 246 nm, 247 nm, 248 nm, 249 nm, or 250 nm, or any combination or range thereof, when measured at 0, 5, 10, 15, 20, 25, 30, 35, or 40 degrees incident light, or between any values therein. In yet further aspects, the filter the filter stack can provide a transmittance of no less than 65%, 70%, 75%, 80%, 85%, or 90% at about 210 nm, 211 nm, 211 nm, 212 nm, 213 nm, 214 nm, 215 nm, 216 nm, 217 nm, 218 nm, 219 nm, 220 nm, 221 nm, 222 nm, 223 nm, 224 nm, 225 nm, 226 nm, 227 nm, 228 nm, 229 nm, 230 nm, 231 nm, 232 nm, 233 nm, 234 nm, 235 nm, 236 nm, 237 nm, 238 nm, 239 nm, 240 nm, 241 nm, 242 nm, 243 nm, 244 nm, 245 nm, 246 nm, 247 nm, 248 nm, 249 nm, or 250 nm, or any combination or range thereof, when measured at 0, 5, 10, 15, 20, 25, 30, 35, or 40 degrees incident light, or between any values therein. For example, the filter stack can provide a transmittance at 222 nm of at least 80%, and all wavelengths in the range of from 215 nm to 230 nm can have a transmittance of at least 65%, when measured at 0 degrees incident light.

The filter stack can provide a stop that reduces transmittance of light at or around 250 nm, 251 nm, 252 nm, 253 nm, 254 nm, 255 nm, 256 nm, 257 nm, 258 nm, 259 nm, or 260 nm, when measured at 0, 5, 10, 15, 20, 25, 30, 35, or 40 degrees incident light. In some aspects, the filter results in reduced, but nonetheless substantial, transmittance of light at one or more wavelength at about 235 nm, 236 nm, 237 nm, 238 nm, 239 nm, 240 nm, 241 nm, 242 nm, 243 nm, 244 nm, 245 nm, 246 nm, 247 nm, 248 nm, 249 nm, 250 nm, 251 nm, 252 nm, 253 nm, 254 nm, 255 nm, 256 nm, 257 nm, 258 nm, 259 nm, or 260 nm. For example, in some aspects, the filter can reduce transmittance at 258 nm, but still provide substantial transmittance at 258 nm. In further examples, the filter can provide a transmittance of less than or about 40%, 35%, 30%, 25%, 20%, 15%, 10%, 5%, 4%, 3%, 2%, 1.5%, 1%, or 0.5% at 245 nm, 246 nm, 247 nm, 248 nm, 249 nm, 250 nm, 251 nm, 252 nm, 253 nm, 254 nm, 255 nm, 256 nm, 257 nm, 258 nm, 259 nm, or 260 nm, or any combination or range thereof, when measured at 0, 5, 10, 15, 20, 25, 30, 35, or 40 degrees incident light. In yet further examples, the filter can provide a transmittance of greater than 20%, 15%, 10%, 5%, 4%, 3%, 2%, 1.5%, 1%, 0.5%, 0.1%, 0.05% or 0.01% at 245 nm, 246 nm, 247 nm, 248 nm, 249 nm, 250 nm, 251 nm, 252 nm, 253 nm, 254 nm, 255 nm, 256 nm, 257 nm, 258 nm, 259 nm, or 260 nm, or any combination or range thereof, when measured at 0, 5, 10, 15, 20, 25, 30, 35, or 40 degrees incident light. In yet a further example, the filter can provide a transmittance of less than 20% at wavelengths between 250 nm and 260 nm, and a transmittance of greater than 5% at 258 nm, when measured at 0 degrees incident light.

In yet further examples, the reduced transmittance provided by the stop can be defined relative to the peak transmittance between 210 nm and 250 nm, when measured at 0, 5, 10, 15, 20, 25, 30, 35, or 40 degrees incident light. For example, the filter stack can provide a transmittance of less than or about 40%, 35%, 30%, 25%, 20%, 15%, 10%, 5%, 4%, 3%, 2%, 1.5%, 1%, or 0.5% relative to peak transmittance at 245 nm, 246 nm, 247 nm, 248 nm, 249 nm, 250 nm, 251 nm, 252 nm, 253 nm, 254 nm, 255 nm, 256 nm, 257 nm, 258 nm, 259 nm, or 260 nm, or any combination or range thereof, when measured at 0, 5, 10, 15, 20, 25, 30, 35, or 40 degrees incident light. In yet further examples, the filter can provide a transmittance of greater than 20%, 15%, 10%, 5%, 4%, 3%, 2%, 1.5%, 1%, or 0.5% relative to peak transmittance at 245 nm, 246 nm, 247 nm, 248 nm, 249 nm, 250 nm, 251 nm, 252 nm, 253 nm, 254 nm, 255 nm, 256 nm, 257 nm, 258 nm, 259 nm, or 260 nm, or any combination or range thereof, when measured at 0, 5, 10, 15, 20, 25, 30, 35, or 40 degrees incident light.

The filter stack can have a 50% cutoff between a region of peak transmittance and a region of minimum transmittance. The 50% cutoff corresponds to a region of 50% transmittance relative to the peak transmittance. The 50% cutoff need not necessarily be a formal cutoff as seen in a one-dimensional shortwave pass filter, but can be a wavelength having 50% transmittance relative to the peak transmittance of a more complex filter design between relative low and high regions. Even in such complex filter designs, it can be helpful to refer to a 50% cutoff for the purposes of comparing to transmittance curves between various filter designs. In various examples, the 50% cutoff can be at or around 230 nm, 231 nm, 232 nm, 233 nm, 234 nm, 235 nm, 236 nm, 237 nm, 238 nm, 239 nm, 240 nm, 241 nm, 242 nm, 243 nm, 244 nm, 245 nm, 246 nm, 247 nm, 248 nm, 249 nm, 250 nm, 251 nm, 252 nm, 253 nm, 254 nm, 255 nm, 256 nm, 257 nm, 258 nm, 259 nm, or 260 nm, measured relative to a peak transmittance between 210 nm and 240 nm, when measured at 0, 5, 10, 15, 20, 25, 30, 35, or 40 degrees incident light. For example, in some aspects, the filter stack can be configured to reduce transmittance of 258 nm light, but preserve transmittance of light at or around 235 nm, 236 nm, 237 nm, 238 nm, 239 nm, 240 nm, 241 nm, 242 nm, 243 nm, 244 nm, 245 nm, 246 nm, 247 nm, 248 nm, 249 nm, or 250 nm, when measured at 0 degrees incident light. The filter stack can have a steep cutoff curve or a gentle cutoff curve. In some aspects, the filter stack can have a 50% cutoff relative to the transmittance at 222 nm. The 50% cutoff relative to 222 nm can be at or around 235 nm, 236 nm, 237 nm, 238 nm, 239 nm, 240 nm, 241 nm, 242 nm, 243 nm, 244 nm, 245 nm, 246 nm, 247 nm, 248 nm, 249 nm, 250 nm, 251 nm, 252 nm, 253 nm, 254 nm, 255 nm, 256 nm, 257 nm, 258 nm, 259 nm, or 260 nm, measured relative to the transmittance at 222 nm, when measured at 0, 5, 10, 15, 20, 25, 30, 35, or 40 degrees incident light. Various aspects can be thus configured to reduce transmittance of 258 nm light, but preserve transmittance of light at or around 235 nm, 236 nm, 237 nm, 238 nm, 239 nm, 240 nm, 241 nm, 242 nm, 243 nm, 244 nm, 245 nm, 246 nm, 247 nm, 248 nm, 249 nm, or 250 nm, when measured at 0 degrees incident light. The filter stack can have a steep cutoff curve or a gentle cutoff curve. For example, in various aspects, the range of wavelengths required for a transition from 80% transmittance relative to a peak transmittance to 20% transmittance relative to a peak transmittance can be 2 nm, 3 nm, 4 nm, 5 nm, 6 nm. 7 nm, 8 nm, 9 nm, 10 nm, 11 nm, 12 nm, 13 nm, 14 nm, or 15 nm.

The filter stack can be configured to provide a pass so as to permit greater transmittance at wavelengths around 260 nm or longer, whereas there is an attenuation of transmittance at around 254 nm. In various aspects, the filter stack can provide a transmittance greater than, or about, 5%, 10%, 15%, 20%, 25%, 30%, 40%, 50%, 60%, or 70%, at about 260 nm, 265 nm, 270 nm, 275 nm, 280 nm, 285 nm, 290 nm, 295 nm, or 300 nm, or any combination or range thereof, when measured at 0, 5, 10, 15, 20, 25, 30, 35, or 40 degrees incident light. For example, the filter stack can provide a transmittance of about 50% or more at one or more wavelengths between 260 nm and 300 nm, as measured at an incident angle of zero degrees.

In various examples, the filter stack is configured to provide a band stop centered around 249 nm, 250 nm, 251 nm, 252 nm, 253 nm, 254 nm, 255 nm, 256 nm, 257 nm, 258 nm, 259 nm, 260 nm, 261 nm, 262 nm, 263 nm, 264 nm, or 265 nm. The band stop can have a 50% shortwave cutoff and a 50% longwave cutoff that are each within about 3 nm, 4 nm, 5 nm, 6 nm. 7 nm, 8 nm, 9 nm, 10 nm, 11 nm, 12 nm, 13 nm, 14 nm, 15 nm, 16 nm, 17 nm, 18 nm, 19 nm, or 20 nm from the center of the band stop. In various aspects, the band stop is not configured to completely attenuate wavelengths within its band stop, but is instead configured to provide at least substantial transmittance of radiation having one or more wavelength 241 nm or longer, 242 nm or longer, 243 nm or longer, 244 nm or longer, 245 nm or longer, 246 nm or longer, 247 nm or longer, 248 nm or longer, 249 nm or longer, 250 nm or longer, 255 nm or longer, 260 nm or longer, 265 nm or longer, 270 nm or longer, 275 nm or longer, or 280 nm or longer.

The filter stack can have a transmittance at 222 nm that is greater off-axis than on-axis. For example, the filter stack can have a 222 nm transmittance measured at an incident angle of zero degrees that is less than a 222 nm transmittance measured at an incident angle of 5, 10, 15, 20, 25, 30, 35, or 40 degrees. In yet further aspects, the filter stack provides a transmittance of at least 80% at one or more wavelength between 225-245, as measured at an incident angle of zero degrees.

The filter stack can have a transmittance at 258 nm that is greater on-axis than off-axis. For example, the filter stack can have a 258 nm transmittance measured at an incident angle of zero degrees that is greater than a 258 nm transmittance measured at an incident angle of 5, 10, 15, 20, 25, 30, 35, or 40 degrees. In yet further aspects, the filter stack can provide a transmittance of at least, or greater than, 0.5%, 1%, 1.5%, 2%, 3%, 4%, 5%, 10%, 15%, 20%, or 25%, at one or more wavelength between 250-260, as measured at an incident angle of zero degrees. In yet further aspects, the filter stack provides a transmittance of less than 0.5%, 1%, 1.5%, 2%, 3%, 4%, 5%, 10%, 15%, 20%, or 25%, at one or more wavelength between 255-260, as measured at an incident angle of zero degrees.

In various further examples, the filter stack provides a peak transmittance at one or more wavelength between 210 nm and 250 nm, a transmittance at one or more wavelengths between 241 nm and 250 nm that is about 90% or more of the peak transmittance, a transmittance at one or more wavelengths between 241 nm and 250 nm that is about 50% of the peak transmittance, and a transmittance at one or more wavelengths between 250 nm and 260 nm that is about 20% or less of the peak transmittance, as measured at an incident angle of 30 degrees. In yet further examples, the filter stack provides a transmittance of at least 80% at 222 nm, as measured at an incident angle of 0, 5, 10, 15, 20, 25, 30, 35, or 40 degrees.

The filter stack, or an far-UV apparatus utilizing it, can provide a transmittance of at least, or greater than, 0.5%, 1%, 1.5%, 2%, 3%, 4%, 5%, 10%, 15%, 20%, or 25% at 254 nm, as measured at an incident angle of zero degrees. In various examples, the filter stack provides a transmittance of at least, or greater than, 0.5%, 1%, 1.5%, 2%, 3%, 4%, 5%, 10%, 15%, 20%, or 25% at 254 nm, as measured at an incident angle of zero degrees, and a total irradiance within a safe ACGIH exposure limit for the eyes or skin, based on an 8, 12, or 24 hour exposure, at a position of expected human exposure or 10 feet from the far-UV radiation source.

The filter stack, or an far-UV apparatus utilizing it, can provide a transmittance of at least, or greater than, 0.5%, 1%, 1.5%, 2%, 3%, 4%, 5%, 10%, 15%, 20%, or 25% at 254 nm, relative to 222 nm transmittance or a peak transmittance between 210-240 nm, and as measured at an incident angle of zero degrees. In various examples, the filter stack provides a transmittance of at least, or greater than, 0.5%, 1%, 1.5%, 2%, 3%, 4%, 5%, 10%, 15%, 20%, or 25% at 254 nm, relative to 222 nm transmittance or a peak transmittance between 210-240 nm, as measured at an incident angle of zero degrees, and a total irradiance within a safe ACGIH exposure limit for the eyes or skin, based on an 8, 12, or 24 hour exposure, at a position of expected human exposure or 10 feet from the far-UV radiation source.

The filter stack includes a low index dielectric material. The low index dielectric material can be a metal oxide-based composition or a fluoride compound. Examples of low index dielectric material include SiO₂, MgF₂, and GdF₃. In various examples, at least one of the low index dielectric material layers is SiO₂. In further examples, in further examples, each of the low index dielectric material layers is SiO₂. The low index dielectric material can have a refractive index of from 1.45 to 1.55 at 222 nm.

The filter stack includes a high index dielectric material. The high index dielectric material can be a metal oxide-based composition. Examples of high index dielectric material include HfO₂, Sc₂O₃, and Al₂O₃. In various examples, at least one of the high index dielectric material layers is HfO₂. In further examples, each of the high index dielectric material layers is HfO₂. The high index dielectric material can have a refractive index of from 2.1 to 2.4 at 222 nm.

Additional suitable dielectric materials can be determined by refractive index at the relevant UV wavelengths, durability, and stability in the presence of UV and/or thermal radiation. See, for example, Palik et al., Handbook of Optical Constants of Solids, 1997, the contents of which are incorporated herewith by reference in its entirety.

In additional aspects, the optical filter stack can include layers, including between high index layers and low index layers that serves to provide a reflectance curve. In various such example, the optical filter stack can be capable of providing a device that efficiently transmits far-UV light from the far-UV radiation source to the treatment space, while also serving to reflect visible light received from the treatment space back out to the treatment space.

Materials used in the optical filter stack can also be selected based on their durability and stability, particularly to UV radiation and heat energy, as well as their cost and ease of use.

In various examples, the first layer that receives incident light is a low index dielectric material. In various other examples, the first layer that receives incident light is a low index dielectric material. In yet further examples, the last layer through which incident light passes is a low index dielectric material. In various other examples, the last layer through which incident light passes is a low index dielectric material.

The optical filter stack can be disposed on a transparent substrate. The transparent substrate can have an internal transmittance at 222 nm of at least 99%. The transparent substrate can provide rigidity and support for the filter stack. It can also be useful for integrating the filter stack with a UV-generating lamp. In some aspects, the transparent substrate can be useful for manufacturing the filter stack. The transparent substrate can be planar or curved. The transparent substrate can be a window, a lens, or an external envelope of a lamp bulb. The transparent substrate can be a crystalline material such as quartz, or a glass such as fused silica. Yet further examples of a transparent substrate include CORNING® 7980 and HERAUS SUPRASIL®. Further examples of suitable transparent substrates include JGS1 and JGS2 optical fused quartz glass.

The optical filter stacks of the present disclosure can be prepared by one or more of sputtering, electron beam evaporation, pulse ion deposition, plasma ion-assisted deposition (PIAD), ion-assisted deposition (IAD) electron beam or thermal evaporation, or other techniques suitable for thin-film coating. The optical filter stacks of the present disclosure can also be prepared by atomic layer deposition (ALD). In various such techniques, non-oxidized Hf and non-oxidized Si are deposited onto the substrate and subsequently oxidized. In various aspects, the filter stack is prepared by plasma ion assisted deposition (PIAD)

Many techniques are understood by those of ordinary skill in the art and various processes can be used to readily obtain the presently described optical filter stacks. See, for example, H. Angus Macleod, Thin-Film Optical Filters, 3rd ed. 2001. Taylor and Francis Group, New York, NY.

In particular examples, the optical filter stack can have a plurality of layers alternating between a high index dielectric material and a low index dielectric material each having an optical thickness configured to together provide a transmittance of at least or about 70%, 75%, 80%, 85%, or 90% at 222 nm; one or more, or each, wavelength selected from 230 nm, 231 nm, 232 nm, 233 nm, 234 nm, 235 nm, 236 nm, 237 nm, 238 nm, 239 nm, 240 nm, 241 nm, 242 nm, 243 nm, 244 nm, and 245 nm having a transmittance about the same or greater than the transmittance at 222 nm; and one or more, or each, wavelength from 255 nm, 256 nm, 257 nm, 258 nm, 259 nm, 260 nm, 261 nm, 262 nm, 263 nm, 264 nm, and 265 nm has a transmittance of less than 15% of the transmittance at 222 nm.

An optical filter stack can have a plurality of layers of a high refractive index dielectric material and layers a low refractive index dielectric material each having an optical thickness configured to together provide a transmittance of at least or about 70%, 75%, 80%, 85%, or 90% at 222 nm; a transmittance at one or more, or each, wavelength selected from 240 nm, 241 nm, 242 nm, 243 nm, 244 nm, and 245 nm that is about 85%, 90%, or 95% or more of a peak transmittance at about 221 nm, 222 nm, 223 nm, 224 nm, 225 nm, 226 nm, 227 nm, 228 nm, 229 nm, 230 nm, 231 nm, 232 nm, 233 nm, 234 nm, 235 nm, 236 nm, 237 nm, 238 nm, 239 nm, 240 nm, 241 nm, 242 nm, 243 nm, 244 nm, or 245 nm; and a transmittance at one or more wavelengths, or each, selected from 255 nm, 256 nm, 257 nm, 258 nm, 259 nm, 260 nm, 261 nm, 262 nm, 263 nm, 264 nm, and 265 nm that is about 1%, 2%, 3%, 4%, 5%, 6%, 7%, 8%, 9%, 10%, 11%, 12%, 13%, 14%, and 15% or less of the peak transmittance as measured at an incident angle of zero degrees.

An optical filter stack can have a plurality of layers of a high refractive index dielectric material and layers a low refractive index dielectric material each having an optical thickness configured to together provide a transmittance of at least 70%, 75%, 80%, 85%, or 90% at 222 nm; a transmittance at one or more, or each, wavelength selected from 242 nm, 243 nm, 244 nm, 245 nm, 246 nm, 247 nm, 248 nm, 249 nm, 250 nm, 251 nm, or 252 nm, that is at least 30%, 35%, 40%, 45% or 50% of a peak transmittance at about 221 nm, 222 nm, 223 nm, 224 nm, 225 nm, 226 nm, 227 nm, 228 nm, 229 nm, 230 nm, 231 nm, 232 nm, 233 nm, 234 nm, 235 nm, 236 nm, 237 nm, 238 nm, 239 nm, 240 nm, 241 nm, 242 nm, 243 nm, 244 nm, and 245 nm; and a transmittance at one or more, or each, wavelength selected from 255 nm, 256 nm, 257 nm, 258 nm, 259 nm, 260 nm, 261 nm, 262 nm, 263 nm, 264 nm, and 265 nm that is at least 3%, 4%, 5%, 6%, 7%, 8%, 9%, 10%, 11%, 12%, 13%, 14%, or 15% of the peak transmittance, as measured at an incident angle of zero degrees.

An optical filter stack can have a plurality of layers of a high refractive index dielectric material and layers a low refractive index dielectric material each having an optical thickness configured to together provide a transmittance of at least 70%, 75%, 80%, 85%, or 90% at 222 nm; a transmittance at one or more, or each, wavelength selected from 255 nm, 256 nm, 257 nm, 258 nm, 259 nm, 260 nm, 261 nm, 262 nm, 263 nm, 264 nm, and 265 nm that is 3%, 4%, 5%, 6%, 7%, 8%, 9%, 10%, 11%, 12%, 13%, 14%, 15%, or any range of values therein, of a peak transmittance at about 221 nm, 222 nm, 223 nm, 224 nm, 225 nm, 226 nm, 227 nm, 228 nm, 229 nm, 230 nm, 231 nm, 232 nm, 233 nm, 234 nm, 235 nm, 236 nm, 237 nm, 238 nm, 239 nm, 240 nm, 241 nm, 242 nm, 243 nm, 244 nm, and 245 nm; and a transmittance at one or more, or each, wavelength selected from 270 nm, 271 nm, 272 nm, 273 nm, 274 nm, 275 nm, 276 nm, 277 nm, 278 nm, 279 nm, 280 nm, 281 nm, 282 nm, 283 nm, 284 nm, 285 nm, 286 nm, 287 nm, 288 nm, 289 nm, 290 nm, 295 nm, 300 nm, 305 nm, 310 nm, 315 nm, 320 nm, 325 nm, 330 nm, 335 nm, 340 nm, 345 nm, 350 nm, 355 nm, 360 nm, 365 nm, and 370 nm, that is 20%, 25%, 30%, 35%, 40%, 45%, 50%, 55%, 60%, 65%, 70%, 75%, 80%, 85%, or 90% or greater than the peak transmittance, as measured at an incident angle of zero degrees.

An optical filter stack can have a plurality of layers of a high refractive index dielectric material and layers a low refractive index dielectric material each having an optical thickness configured to together provide a transmittance of at least 70%, 75%, 80%, 85%, or 90% at 222 nm; a transmittance at one or more, or each, wavelength selected from 255 nm, 256 nm, 257 nm, 258 nm, 259 nm, 260 nm, 261 nm, 262 nm, 263 nm, 264 nm, and 265 nm that is 3%, 4%, 5%, 6%, 7%, 8%, 9%, 10%, 11%, 12%, 13%, 14%, 15%, or any range of values therein, of a peak transmittance at about 221 nm, 222 nm, 223 nm, 224 nm, 225 nm, 226 nm, 227 nm, 228 nm, 229 nm, 230 nm, 231 nm, 232 nm, 233 nm, 234 nm, 235 nm, 236 nm, 237 nm, 238 nm, 239 nm, 240 nm, 241 nm, 242 nm, 243 nm, 244 nm, and 245 nm; and a transmittance at one or more, or each, wavelength selected from 270 nm, 271 nm, 272 nm, 273 nm, 274 nm, 275 nm, 276 nm, 277 nm, 278 nm, 279 nm, and 280 nm that is 1%, 5%, 10%, 20%, 25%, 30%, 35%, 40%, 45%, 50% or greater than the peak transmittance, as measured at an incident angle of zero degrees.

An optical filter stack can have a plurality of layers of a high refractive index dielectric material and layers a low refractive index dielectric material each having an optical thickness configured to together provide a transmittance of at least 70%, 75%, 80%, 85%, or 90% at 222 nm; a transmittance at one or more, or each, wavelength selected from 230 nm, 231 nm, 232 nm, 233 nm, 234 nm, 235 nm, 236 nm, 237 nm, 238 nm, 239 nm, 240 nm, 241 nm, 242 nm, 243 nm, 244 nm, and 245 nm, that is at least 30%, 40%, or 50% of a peak transmittance from 222 nm, 223 nm, 224 nm, 225 nm, 226 nm, 227 nm, 228 nm, 229 nm, 230 nm, 231 nm, 232 nm, 233 nm, 234 nm, 235 nm; a transmittance at one or more wavelengths, or each, selected from 245 nm, 246 nm, 247 nm, 248 nm, 249 nm, 250 nm, 251 nm, 252 nm, 253 nm, 254 nm, 255 nm, 256 nm, 257 nm, 258 nm, 259 nm, 260 nm that is about 1%, 2%, 3%, 4%, 5%, 6%, 7%, 8%, 9%, 10%, 11%, 12%, 13%, 14%, 15%, or any range of values therein, of the peak transmittance; a transmittance at one or more, or each, wavelength selected from 270 nm, 271 nm, 272 nm, 273 nm, 274 nm, 275 nm, 276 nm, 277 nm, 278 nm, 279 nm, and 280 nm that is 5%, 10%, 15%, 20% or greater than the peak transmittance; and a transmittance at one or more, or each, wavelength selected from 270 nm, 271 nm, 272 nm, 273 nm, 274 nm, 275 nm, 276 nm, 277 nm, 278 nm, 279 nm, 280 nm, 281 nm, 282 nm, 283 nm, 284 nm, 285 nm, 286 nm, 287 nm, 288 nm, 289 nm, 290 nm, 295 nm, 300 nm, 305 nm, 310 nm, 315 nm, 320 nm, 325 nm, 330 nm, 335 nm, 340 nm, 345 nm, 350 nm, 355 nm, 360 nm, 365 nm, and 370 nm, that is 20%, 25%, 30%, 35%, 40%, 45%, 50%, 55%, 60%, 65%, 70%, 75%, 80%, 85%, or 90% or greater than the peak transmittance, as measured at an incident angle of zero degrees.

The disclosure also provides an optical filter stack configured to transmit one or more wavelength of far-UV radiation while reflecting one or more wavelength of visible light. For example, the optical filter stack can have a plurality of layers of a high refractive index dielectric material and layers a low refractive index dielectric material each having an optical thickness configured to together provide a transmittance curve having a peak transmittance at a wavelength between about 215 nm and 235 nm, and a reflectance curve having a reflectance of at least 50% for one or more wavelength of visible light having a wavelength greater than 450. The layers of an optical filter stack can be configured for reflectance in the same manner that they are configured for reducing transmittance. See, for example, H. Angus Macleod, Thin-Film Optical Filters, 3rd ed. 2001. Taylor and Francis Group, New York, NY, which is incorporated by reference herewith in its entirety.

The reflectance curve can have a reflectance of at least or about 50%, 55%, 60%, 65%, 70%, 75%, 80%, 85%, or 90% at one or more wavelength at 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, or longer. The reflectance curve can have a reflectance of at least or about 50%, 55%, 60%, 65%, 70%, 75%, 80%, 85%, or 90% at each wavelength between about 430 nm to 550 nm, 430 nm to 540 nm, 430 nm to 530 nm, 430 nm to 520 nm, 430 nm to 510 nm, 430 nm to 500 nm, 430 nm to 490 nm, 430 nm to 480 nm, 430 nm to 470 nm, 430 nm to 460 nm, 430 nm to 450 nm, or 430 nm to 440 nm.

The reflectance curve can have a reflectance of about or less than about 5%, 10%, 20%, 30%, 40%, or 50%, at one or more wavelength from about 380 nm to about 420 nm. The reflectance curve can have a reflectance of about or less than about 5%, 10%, 20%, 30%, 40%, or 50%, at one or more wavelength from about 390 nm to about 410 nm. The reflectance curve can have a reflectance of about or less than about 5%, 10%, 20%, 30%, 40%, or 50%, at one or more wavelength from about 380 nm to about 420 nm. The reflectance curve can have a reflectance of about or less than about 5%, 10%, 20%, 30%, 40%, or 50%, at one or more wavelength from about 400 nm.

The optical filter stack can be configured to have a reflectance curve that reflects visible light, particularly visible light from the side of the filter stack that is not configured to receive far-UV light. The optical filter stack can be configured to have a reflectance curve that reflects non-purple visible light. The reflectance curve can have low reflectance of purple light and high reflectance of non-purple light.

The optical filter stack can be configured for a reflectance curve having a reflectance of at least 50% at one or more wavelength from about 430 nm to about 750 nm, a reflectance curve having a reflectance of at least 60% at one or more wavelength from about 430 nm to about 750 nm, a reflectance curve having a reflectance of at least 70% at one or more wavelength from about 430 nm to about 750 nm, a reflectance curve having a reflectance of at least 80% at one or more wavelength from about 430 nm to about 750 nm, a reflectance curve having a reflectance of at least 50% for each wavelength from about 430 nm to about 580 nm, a reflectance curve having a reflectance of at least 60% for each wavelength from about 440 nm to about 580 nm, a reflectance curve having a reflectance of at least 60% for each wavelength from about 450 nm to about 580 nm, a reflectance curve having a reflectance of at least 70% for each wavelength from about 460 nm to about 570 nm, a reflectance curve having a reflectance of at least 70% for each wavelength from about 500 nm to about 570 nm, a reflectance curve having a reflectance of at least 80% for each wavelength from about 540 nm to about 560 nm. In various aspects, the reflectance curve is configured for greater reflectance from 440 nm to 580 nm, less reflectance at wavelengths loner than 600 nm, and less reflectance from 360 nm to 400 nm.

One advantage to utilizing reflectance of visible light is that it can visually obscure the purple-pink appearance of far-UV radiation sources, which may take the form of bulbs, emission tube, excimer lamp, etc. For example, a target optical filter stack can transmit far-UV radiation like 222 nm light while reflecting visible light, while reflecting all other wavelengths, while reflecting visible light other than purple or pink light, or reflecting all other wavelengths other than purple or pink light. Example transmittance curves may have a first transmittance peak around about 215 nm to about 240 nm, and a second transmittance peak around about 380 nm to about 700 nm, about 380 nm to about 450 nm, or about 300 nm to about 340 nm. In another aspect, the optical filter stack is configured to transmit one or more wavelengths from about 200 nm to about 260 nm and reflect one or more wavelengths of visible light from about 420 nm to about 680 nm. Visible emissions of UV lamps can be obscured with about 40% ambient light being reflected. In further aspects, the optical filter stack can be configured to transmit one or more, or all, wavelength at about 220 nm to about 230 nm; attenuate one or more, or all, wavelengths below 215 nm; attenuate one or more, or all, wavelengths at about 240 nm to about 280 nm; and/or attenuate one or more, or all, wavelengths above about 340 nm, 350 nm, 360 nm, 370 nm, 380 nm, 390 nm, or 400 nm.

In various aspects, the optical filter stack can contain additional layers with a moderate refractive index less than the high refractive index dielectric material and greater than the low refractive index dielectric material, and such layers can be used in between the high refractive index and low refractive index layers, or in place of one of the high refractive index or low refractive index layers. In various aspects, the optical filter stack can include HfO₂, SiO₂, Sc₂O₃, and Al₂O₃, or any combination thereof. The optical filter stack can include layers that comprise hafnium, silicon, aluminum, or scandium. The optical filter stack can include reflective layers other than the high refractive index and low refractive index layers, and the reflective layer can comprise hafnium, silicon, aluminum, or scandium. The reflective layer can be located between the high refractive index and low refractive index layers, or in place of one of the high refractive index or low refractive index layers.

### Far-UV Apparatus

The presently discussed optical filter stack can be incorporated into an apparatus comprising a far-UV radiation source that generates radiation having one or more wavelengths between about 210 nm to about 230 nm. The radiation is transmitted through a transparent substrate comprising the optical filter stack described herein. The optical filter stack can be disposed directly onto a far-UV generating bulb, or disposed upon a window or lens through which the apparatus emits far-UV radiation. The apparatus can be useful for inactivating pathogens in air.

The apparatus can provide emissions, after transmitting through the filter stack, such that a peak emission (i.e., peak filtered emission), is between 210 nm to 240 nm, such as 222 nm. In various example, the apparatus provides an emission at one or more wavelengths between 237 nm and 245 nm having an intensity of about 1%, 2%, 3%, 4%, 5%, 10%, 15%, 20%, 25%, 30%, 35%, 40%, 45%, or 50%, or more of the peak emission, or any range of values therein, as measured at an incident angle of zero degrees. In another example, the apparatus provides an emission at one or more wavelengths between 241 nm and 260 nm having an intensity of about 0.5%, 1%, 1.5%, 2%, 3%, 4%, 5%, 10%, 15%, 20%, or 25% of the peak emission, or any range of values therein, as measured at an incident angle of zero degrees. In another example, the apparatus provides an emission at one or more wavelengths between 250 nm and 265 nm having an intensity of about 0.1%, 0.5%, 1%, 1.5%, 2%, 3%, 4%, 5%, 10%, 15%, 20%, or 25% of the peak emission, or any range of values therein, as measured at an incident angle of zero degrees.

The far-UV radiation source can utilize various technologies, including one or more bulbs, light-emitting diodes (LEDs), or other sources configured to emit UV radiation in an amount effective to inactivate pathogens in air.

The far-UV radiation source can include one or more excimer bulb. Excimer bulbs include a combination of a rare gas and halogen which emits one or more wavelength of far-UV light when excited by an electrical stimulus. Excimer bulbs excitation can be triggered in various ways including, for example, glow discharge, pulsed discharge, dielectric barrier discharge, short arc, or combinations thereof. In various examples, the far-UV radiation source comprises a KrCl excimer bulb or a KrBr excimer bulb. The far-UV radiation source generates and emits radiation comprising one or more wavelengths of about 210 nm to about 230 nm but may also emit other wavelengths as well. In some examples, the far-UV radiation source can be a source that provides a peak unfiltered emission between 210 nm to 240 nm, such as 222 nm. In various further examples, the far-UV radiation source can emit UV wavelengths other than far-UV. In some examples, it can emit visible light. In further examples, the excimer bulb generates and emits 222 nm light. Various shapes of bulbs are possible. Bulbs are typically substantially tube-like, and can be rod-shaped, oblong, toroidal, or flattened versions of the same. The far-UV radiation source can include a bulb having an annular body having an outer surface and defining an internal discharge cavity, wherein the annular body has a major axial dimension and a minor radial dimension, and comprises an electrode in the internal discharge cavity that traverses along the major axial dimension of the annular body.

Krypton chloride excimer lamps typically emit a peak emission at 221.8 nm, but convention in the art is to refer to such emission as 222 nm.

The far-UV radiation source can include a housing, the form of which is not particularly restrictive. Typically, the far-UV radiation source includes a housing containing one or more bulb. In various aspects, the housing partially encloses the one or more bulb and provides at least one opening through which radiation cam be emitted. For example, the housing can be a box where one face has an opening that is covered by a UV-transparent material serving as a protective window.

The far-UV radiation source can further include any number of optical components in addition to the optical filter stack described herein. For example, the inside of the housing can be furnished with reflectors to direct light out the housing and maximize emitted radiation. The far-UV radiation source can include one or more component for adjusting the direction, intensity, or focus of the emitted light. For example, the far-UV radiation source can include a diffuser or lens.

In various aspects, the apparatus contains a reflector behind the bulbs, emission tube, excimer lamp.

The one or more far-UV radiation source, or an apparatus utilizing it together with an optical filter stack described herein, can irradiate an occupiable space with a fluence rate or irradiance at a value from about 0.5 μW/cm² to about 60 μW/cm² at a distance of 1, 2, 3, 4, 5, 6, 7, 8, 9, or 10 meters from the far-UV radiation source. For example, the fluence rate or irradiance can be about or less than 0.5 μW/cm², 1.0 μW/cm², 1.5 μW/cm², 2.0 μW/cm², 2.5 μW/cm², 3.0 μW/cm², 3.5 μW/cm², 4.0 μW/cm², 4.5 μW/cm², 5.0 μW/cm², 5.5 μW/cm², 6.0 μW/cm², 6.5 μW/cm², 7.0 μW/cm², 7.5 μW/cm², 8.0 μW/cm², 8.5 μW/cm², 9.0 μW/cm², 9.5 μW/cm², 10 μW/cm², 11 μW/cm², 12 μW/cm², 13 μW/cm², 14 μW/cm², 15 μW/cm², 16 μW/cm², 17 μW/cm², 18 μW/cm², 19 μW/cm², 20 μW/cm², 21 μW/cm², 22 μW/cm², 23 μW/cm², 24 μW/cm², 25 μW/cm², 26 μW/cm², 27 μW/cm², 28 μW/cm², 29 μW/cm², 30 μW/cm², 31 μW/cm², 32 μW/cm², 33 μW/cm², 34 μW/cm², 35 μW/cm², 36 μW/cm², 37 μW/cm², 38 μW/cm², 39 μW/cm², 40 μW/cm², 41 μW/cm², 42 μW/cm², 43 μW/cm², 44 μW/cm², 45 μW/cm², 46 μW/cm², 47 μW/cm², 48 μW/cm², 49 μW/cm², 50 μW/cm², 51 μW/cm², 52 μW/cm², 53 μW/cm², 54 μW/cm², 55 μW/cm², 56 μW/cm², 57 μW/cm², 58 μW/cm², 59 μW/cm², or 60 μW/cm², or any range of values therein, at a distance of 1 meter from the far-UV radiation source or at a nearest distance between the far-UV radiation source and an location of an expected occupant. In some aspects, the one or more far-UV radiation source can irradiate an occupiable space with 10 μW/cm², 11 μW/cm², 12 μW/cm², 13 μW/cm², 14 μW/cm², or 15 μW/cm² at a distance of 1 meter from the far-UV radiation source or at a nearest distance between the far-UV radiation source and a location of an expected occupant

The one or more far-UV radiation source can irradiate an occupiable space with a total optical power flux of about or less than 4 mW, 5 mW, 6 mW, 7 mW, 8 mW, 9 mW, 10 mW, 15 mW, 20 mW, 25 mW, 30 mW, 35 mW, 40 mW, 45 mW, 50 mW, 55 mW, 60 mW, 65 mW, 70 mW, 75 mW, 80 mW, 85 mW, 90 mW, 95 mW, 100 mW, 105 mW, 110 mW, 115 mW, 120 mW, 125 mW, 130 mW, 135 mW, 140 mW, 145 mW, 150 mW, 155 mW, 160 mW, 165 mW, 170 mW, 175 mW, 180 mW, 185 mW, 190 mW, 195 mW, 200 mW, 205 mW, 210 mW, 215 mW, 220 mW, 225 mW, 230 mW, 235 mW, 240 mW, 245 mW, 250 mW, 255 mW, 260 mW, 265 mW, 270 mW, 275 mW, 280 mW, 285 mW, 290 mW, 295 mW, 300 mW, 305 mW, 310 mW, 315 mW, 320 mW, 325 mW, 330 mW, 335 mW, 340 mW, 345 mW, 350 mW, 355 mW, 360 mW 15 mW, 20 mW, 25 mW, 30 mW, 35 mW, 40 mW, 45 mW, 50 mW, 55 mW, 60 mW, 65 mW, 70 mW, 75 mW, 80 mW, 85 mW, 90 mW, 95 mW, 100 mW, 101 mW, 102 mW, 103 mW, 104 mW, 105 mW, 106 mW, 107 mW, 108 mW, 109 mW, 110 mW, 111 mW, 112 mW, 113 mW, 114 mW, 115 mW, 116 mW, 117 mW, 118 mW, 119 mW, 120 mW, 121 mW, 122 mW, 123 mW, 124 mW, 125 mW, 126 mW, 127 mW, 128 mW, 129 mW, 130 mW, 131 mW, 132 mW, 133 mW, 134 mW, 135 mW, 136 mW, 137 mW, 138 mW, 139 mW, 140 mW, 145 mW, 150 mW, 155 mW, 160 mW, 165 mW, 170 mW, 175 mW, 180 mW, 185 mW, 190 mW, 195 mW, 200 mW, 205 mW, 210 mW, 215 mW, 220 mW, 225 mW, 230 mW, 235 mW, 240 mW, 245 mW, 250 mW, 255 mW, 260 mW, 265 mW, 270 mW, 275 mW, 280 mW, 285 mW, 290 mW, 295 mW, 300 mW, 305 mW, 310 mW, 315 mW, 320 mW, 325 mW, 330 mW, 335 mW, 340 mW, 345 mW, 350 mW, 355 mW, or 360 mW, or any range of values therein. For example, the one or more far-UV radiation source can irradiate an occupiable space with a total optical power flux of 120 mW.

The apparatus can be configured, particularly by use of the optical filter stack or an optical utilizing the same, such that germicidal wavelengths are transmitted while harmful wavelengths are controlled. For example, the filter can attenuate wavelengths that are harmful to human eye and skin tissue. In various examples, the filter is configured such that when used in an apparatus that generates far-UV light, it results in radiation upon human eyes or skin of less than 50 mJ/cm² at a wavelength of 230 nm and less than 10 mJ/cm² between a wavelength of 240 and 280 nm, when measured at its intended distance from a person, or as measured at 1, 2, 3, 4, 5, 6, 7, 8, 9, or 10 meters, over a period of exposure, instantaneous exposure, or 1 s. As a further example, the apparatus can be configured such that the emitted radiation is about 0.5 μW/cm² to about 60 μW/cm² at one or more occupant over a period of occupancy, or at one or more area of expected occupancy over a period of expected occupancy. The far-UV apparatus can be configured such that when deployed in an occupied space, the emitted radiation lacks the intensity, power, fluence, fluence rate, irradiance, effective irradiance, or total irradiance necessary for inactivating pathogens on the skin or other surface of an occupant. The far-UV apparatus can be configured such that when deployed in an occupied space, the emitted radiation lacks the intensity, power, fluence, fluence rate, irradiance, effective irradiance, or total irradiance necessary for inactivating pathogens on objects or surfaces in the treatment space.

The apparatus can also be configured such that the transparent substrate and the filter stack receive radiation at an incident angle of zero degrees to 40 degrees. In various examples, the majority of incident light is at zero degrees. Yet, in other examples, the majority of incident light is off-axis, at an incident angle of at least, or greater than, 0, 5, 10, 15, 20, 25, 30, 35, or 40 degrees.

In a further example, the apparatus is configured such that more than 50%, 60%, 70%, 80%, 85%, 90%, or 95% of the radiation incident to the transparent substrate and the filter stack has an angle of at least, or greater than, 0, 5, 10, 15, 20, 25, 30, 35, or 40 degrees.

The apparatus for inactivating a pathogen in air can include a far-UV radiation source that emits radiation through a window comprising an optical filter stack having plurality of layers of a high refractive index dielectric material and layers a low refractive index dielectric material each having an optical thickness configured to together provide a peak emission at about 222 nm, and an emission at one or more wavelengths from 250 nm to 265 nm having an intensity of 0.1% or more of the emission at 222 nm, as measured upon treatment with an unfiltered KrCl excimer emission, as measured at an incident angle of zero degrees. In further aspects, the emissions can include one or more wavelengths from 237 nm to 245 nm having an intensity of 1% or more of its emission at 222 nm. In yet further aspects, the emissions can include one or more wavelengths between 241 nm and 245 nm having an intensity of 1% or more of the emission at 222 nm, and an emission at 258 nm having an intensity of 0.1% or more of the emission at 222 nm, as measured upon treatment with an unfiltered KrCl excimer emission, as measured at an incident angle of zero degrees.

The apparatus and optical filter stack can be configured for off-axis light. For example, the apparatus can have a window coated with the presently described optical filter, wherein the window is configured to receive radiation at an incident angle of zero degrees to at least 30 degrees. In further examples, more than 80% of the radiation incident to the window has an angle of greater than 20 degrees. The window can be substantially planar, or it can be curved. The window can be constructed of the same material as the substrate upon which the optical filter stack is coated, or the substrate coated with the optical filter stack itself can serve as the window. For example, the window can be a crystal such as quartz, or a glass such as fused silica. In some aspects, the window can be CORNING® 7980 or HERAUS SUPRASIL®. Further examples of suitable transparent substrates include JGS1 and JGS2 optical fused quartz glass.

The apparatus can be used in a method of inactivating a pathogen in air. A method for inactivating a pathogen in air, comprising irradiating air in a human-use space with UV radiation having a peak emission at 222 nm, an emission at one or more wavelengths from 237 nm to 245 nm having an intensity of 1% or more of the emission at 222 nm, and an emission at one or more wavelengths from 250 nm to 265 nm having an intensity of 0.1% or more of the emission at 222 nm. The UV radiation can have an emission at one or more, or each, of 240 nm, 241 nm, 242 nm, 243 nm, 244 nm, or 245 nm having an intensity of 1% or more of the emission at 222 nm. The UV radiation can have an emission at 241 nm having an intensity of 1% or more of the emission at 222 nm. The UV radiation can have an emission at one or more, or each, wavelength selected from 250 nm, 251 nm, 252 nm, 253 nm, 254 nm, 255 nm, 256 nm, 257 nm, 258 nm, 259 nm, 260 nm, 261 nm, 262 nm, 263 nm, 264 nm, or 265 nm, having an intensity of 0.1%, 0.2%, 0.3%, 0.4%, 0.5%, 0.6%, 0.7%, 0.8%, 0.9%, 1%, 2%, 3%, 4%, 5%, or more of the emission at 222 nm.

The various aspects described herein are not intended to be construed as exclusive or specific. Rather, a person of ordinary skill would appreciate that combinations and permutations of the aforementioned aspects are within the scope of invention. The present invention can be further understood by reference to the following Examples which are offered by way of illustration. The present invention is not limited to the Examples given herein.

### EXAMPLES

### Example 1

An optical filter stack was designed that largely passes far-UV light having (1) a wavelength of 242 nm or shorter and (2) a wavelength of 277 nm and longer. The filter stack was also designed to transmit at least 4% of each wavelength between 240 nm and 280 nm, relative to a peak transmittance between 210 nm and 250 nm as measured at an incident angle of zero degrees. The peak transmittance between 210 nm and 250 nm is modeled to occur at about 239 nm. The filter stack also a first longwave peak between 270 nm and 300 nm that is around 282 nm, and a second longwave peak that is between 300 nm and 350 nm that is around 317 nm.

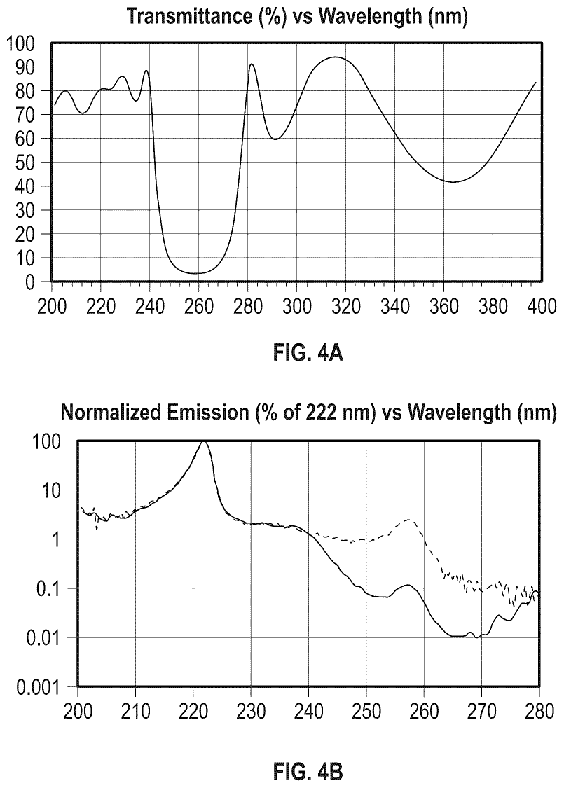

Example 0 is designed to provide about 90% of peak transmittance at about 240 nm, about 75% of peak transmittance at about 241 nm, about 50% of peak transmittance between about 242 nm to 243 nm, about 25% of peak transmittance at about 245 nm, about 10% of peak transmittance at about 249 nm, and a transmittance between 245 and 270 of no less than 4% of peak transmittance, for light having an incident angle of zero degrees. The filter stack also has additional peaks in longer wavelengths. Specifically, a first longwave peak at about 282 nm is about the same or greater than the shortwave peak at about 239 nm, and a second longwave peak at about 317 nm is about the same or greater than the shortwave peak at about 239 nm. A chart illustrating transmittance is provided in FIG. 4A. Transmittance of light having an incident angle of zero degrees is shown by the solid line.

The physical construction of layers is provided in Table 0. The substrate is an amorphous fused silica (7980 Standard) upon which the alternating layers of high index of refraction (HfO₂) and low index of refraction (SiO₂) are disposed. The filter stack has 16 layers and a total thickness of 979.22 nm.

TABLE 1
Filter stack according to Example 1.

| | Material | Physical |
|---|---|---|
| Layer | 7980 | Thickness |
| Substrate | Standard | (nm) |
| 1 | HfO2 | 80.27 |
| 2 | SiO2 | 68.78 |
| 3 | HfO2 | 18.01 |
| 4 | SiO2 | 137.8 |
| 5 | HfO2 | 17.6 |
| 6 | SiO2 | 75.04 |
| 7 | HfO2 | 15.16 |
| 8 | SiO2 | 65.62 |
| 9 | HfO2 | 69.73 |
| 10 | SiO2 | 63.69 |
| 11 | HfO2 | 18.02 |
| 12 | SiO2 | 70.72 |
| 13 | HfO2 | 17.03 |
| 14 | SiO2 | 141.68 |
| 15 | HfO2 | 20.87 |
| 16 | SiO2 | 99.19 |
| Medium | Air | |

| | |
|---|---|
| Total Thickness | 979.22 |

Example 1 was evaluated to determine how the optical filter stack would affect emissions of far-UV light, particularly light generated by a KrCl excimer bulb. The optical filter stack according to Example 1 is modeled to provide a peak emission at 221.9 nm for light having an incident angle of zero degrees, an emission at 240 nm having an intensity of 1.26% of the peak emission, an emission at 241 nm having an intensity of 1.05% of the peak emission, an emission at 250 nm having an intensity of 0.08% of the peak emission, and an emission at 258 nm having an intensity of 0.11% of the peak emission. A 1% emission relative to the peak emission at 221.9 nm is reached at 241.06 nm. FIG. 4B provides a chart illustrating the emission profile of Example 1 (black) in comparison with unfiltered emissions from a KrCl excimer bulb (gray).

### Example 2

An optical filter stack was designed that largely passes far-UV light having (1) a wavelength of 243 nm or shorter and (2) a wavelength of 279 nm and longer. The filter stack was also designed to transmit at least 12% of each wavelength between 240 nm and 280 nm, relative to a peak transmittance between 210 nm and 250 nm as measured at an incident angle of zero degrees. The peak transmittance is modeled to occur at about 238 nm.

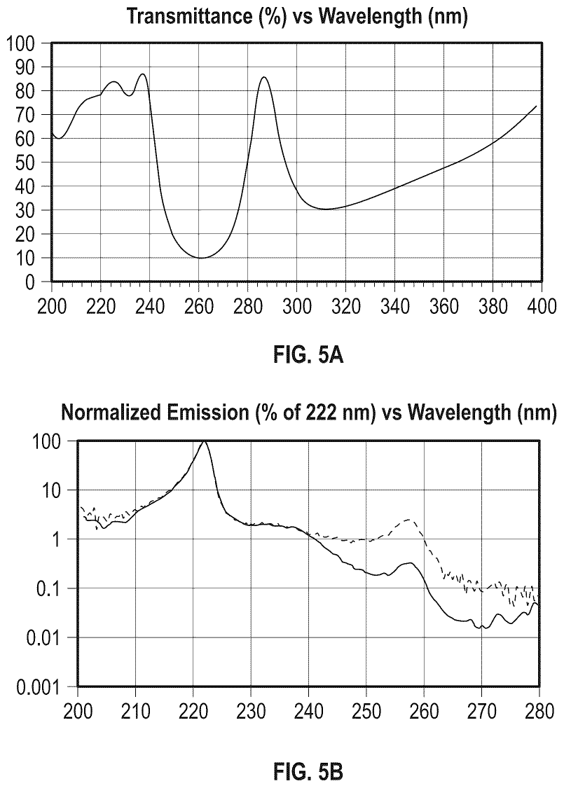

Example 0 is designed to provide about 90% of peak transmittance at about 239.5 nm, about 75% of peak transmittance at about 241 nm, about 50% of peak transmittance at about 243.5 nm, about 25% of peak transmittance at about 248.5 nm, about 20% of peak transmittance at about 250.5 nm, and a transmittance between 245 and 270 of no less than 12% of peak transmittance, for light having an incident angle of zero degrees. The filter stack also has an additional peak in longer wavelengths, namely, a longwave peak at about 287 nm that has a transmittance that is about the same as the shortwave peak at about 238 nm. A chart illustrating transmittance is provided in FIG. 5A. Transmittance of light having an incident angle of zero degrees is shown by the solid line.

The physical construction of layers is provided in Table 2. The substrate is an amorphous fused silica (7980 Standard) upon which the alternating layers of high index of refraction (HfO₂) and low index of refraction (SiO₂) are disposed. The filter stack has 12 layers and a total thickness of 624.44 nm.

TABLE 2
Filter stack according to Example 2.

| | | |
|---|---|---|
| | | Physical |
| Layer | Material | Thickness (nm) |

| | |
|---|---|
| Substrate | 7980 Standard |

| | | |
|---|---|---|
| 1 | HfO2 | 83.04 |
| 2 | SiO2 | 53.79 |
| 3 | HfO2 | 36.73 |
| 4 | SiO2 | 36.18 |
| 5 | HfO2 | 50.51 |
| 6 | SiO2 | 86.52 |
| 7 | HfO2 | 32.59 |
| 8 | SiO2 | 50.89 |
| 9 | HfO2 | 33.88 |
| 10 | SiO2 | 46.19 |
| 11 | HfO2 | 39.8 |
| 12 | SiO2 | 74.32 |
| Medium | Air | |

| | |
|---|---|
| Total Thickness | 624.44 |

Example 2 was evaluated to determine how the optical filter stack would affect emissions of far-UV light, particularly light generated by a KrCl excimer bulb. The optical filter stack according to Example 2 is modeled to provide a peak emission at 221.9 nm for light having an incident angle of zero degrees, an emission at 240 nm having an intensity of 1.17% of the peak emission, an emission at 241 nm having an intensity of 1.03% of the peak emission, an emission at 250 nm having an intensity of 0.21% of the peak emission, and an emission at 258 nm having an intensity of 0.31% of the peak emission. A 1% emission relative to the peak emission at 221.9 nm is reached at 241.04 nm. FIG. 5B provides a chart illustrating the emission profile of Example 2 (black) in comparison with unfiltered emissions from a KrCl excimer bulb (gray).

### Example 3

An optical filter stack was designed that largely passes far-UV light having (1) a wavelength of 248 nm or shorter and (2) a wavelength of 317 nm and longer. The filter stack was also designed to transmit at least 1.4% of each wavelength between 240 nm and 280 nm; at least 5% of each wavelength between 250 nm and 259 nm; and at least 10% of each wavelength between 240 nm and 255, each relative to a peak transmittance between 210 nm and 245 nm as measured at an incident angle of zero degrees. The peak transmittance is modeled to occur at about 242 nm.

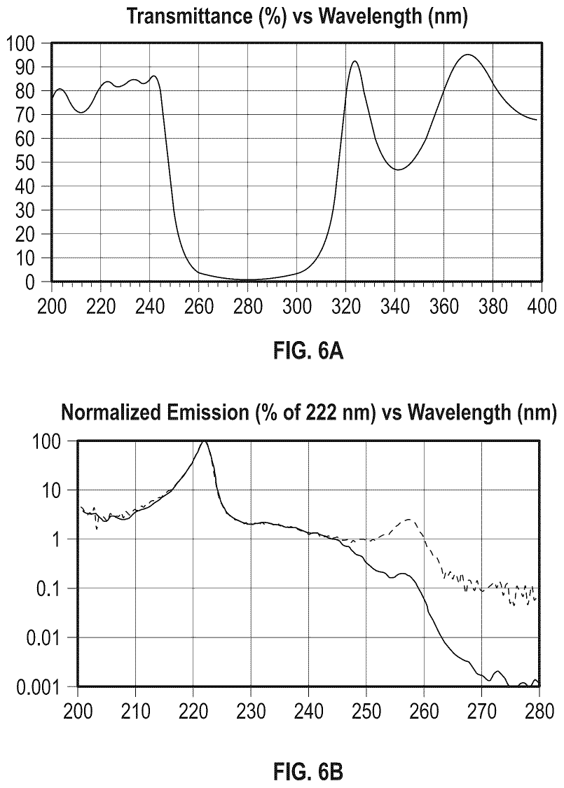

Example 3 is designed to provide about 90% of peak transmittance at about 244.5 nm, about 75% of peak transmittance at about 246 nm, about 50% of peak transmittance at about 248.5 nm, about 25% of peak transmittance at about 251.5 nm, about 10% of peak transmittance at about 255.5 nm, and a transmittance between 245 and 270 of no less than 1.75% of peak transmittance, for light having an incident angle of zero degrees. The filter stack also has an additional peak in longer wavelengths, namely, a longwave peak at about 324 nm that has a transmittance that is about the same or greater than the shortwave peak at about 242 nm. A chart illustrating transmittance is provided in FIG. 6A. Transmittance of light having an incident angle of zero degrees is shown by the solid line.

The physical construction of layers is provided in Table 0. The substrate is an amorphous fused silica (7980 Standard) upon which the alternating layers of high index of refraction (HfO₂) and low index of refraction (SiO₂) are disposed. The filter stack has 15 layers and a total thickness of 606.3 nm.

TABLE 3
Filter stack according to Example 3.

| | | |
|---|---|---|
| | | Physical |
| Layer | Material | Thickness (nm) |

| | |
|---|---|
| Substrate | 7980 Standard |

| | | |
|---|---|---|
| 1 | HfO2 | 35.9 |
| 2 | SiO2 | 51.17 |
| 3 | HfO2 | 35.9 |
| 4 | SiO2 | 44.15 |
| 5 | HfO2 | 30.54 |
| 6 | SiO2 | 49.85 |
| 7 | HfO2 | 29.39 |
| 8 | SiO2 | 49.69 |
| 9 | HfO2 | 31.19 |
| 10 | SiO2 | 47.97 |
| 11 | HfO2 | 33.62 |
| 12 | SiO2 | 45.07 |
| 13 | HfO2 | 34.56 |
| 14 | SiO2 | 46.52 |
| 15 | HfO2 | 40.79 |
| Medium | Air | |

| | |
|---|---|
| Total Thickness | 606.3 |

Example 3 was evaluated to determine how the optical filter stack would affect emissions of far-UV light, particularly light generated by a KrCl excimer bulb. The optical filter stack according to Example 3 is modeled to provide a peak emission at 221.9 nm for light having an incident angle of zero degrees, an emission at 240 nm having an intensity of 1.28% of the peak emission, an emission at 241 nm having an intensity of 1.30% of the peak emission, an emission at 242 nm having an intensity of 1.18% of the peak emission, an emission at 243 nm having an intensity of 1.10% of the peak emission, an emission at 250 nm having an intensity of 0.31% of the peak emission, and an emission at 258 nm having an intensity of 0.15% of the peak emission. A 1% emission relative to the peak emission at 221.9 nm is reached at 243.76 nm. FIG. 6B provides a chart illustrating the emission profile of Example 3 (black) in comparison with unfiltered emissions from a KrCl excimer bulb (gray).

### Example 4

An optical filter stack was designed that largely passes far-UV light having (1) a wavelength of 244.5 nm or shorter and (2) a wavelength of 277.5 nm and longer. The filter stack was also designed to transmit at least 1% of each wavelength between 240 nm and 280 nm; at least 5% of each wavelength between 240 nm and 250 nm; and at least 10% of each wavelength between 274 nm and 280, each relative to a peak transmittance between 210 nm and 245 nm as measured at an incident angle of zero degrees. The peak transmittance is modeled to occur at about 242 nm.

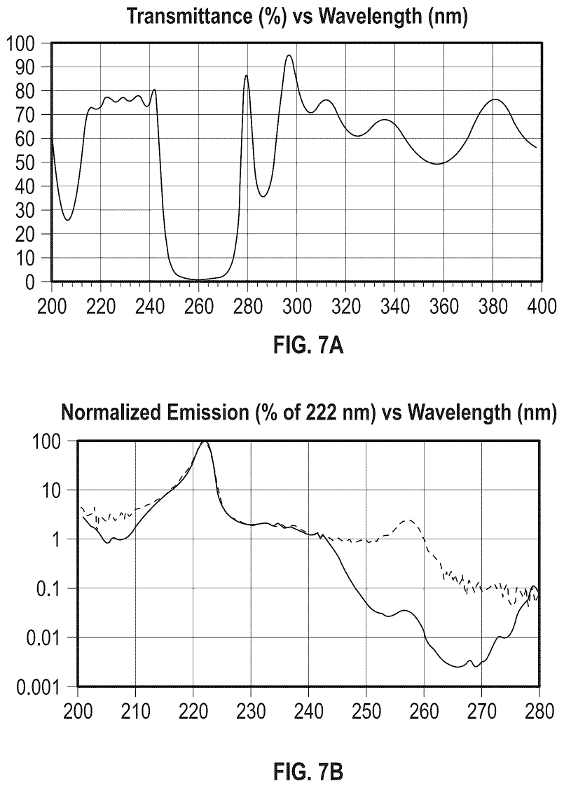

Example 4 is designed to provide about 90% of peak transmittance at about 243 nm, about 75% of peak transmittance at about 243.5 nm, about 50% of peak transmittance at about 244.5 nm, about 25% of peak transmittance at about 245.5 nm, about 10% of peak transmittance at about 248.5 nm, and a transmittance between 250 and 270 of no less than 1% of peak transmittance, for light having an incident angle of zero degrees. The filter stack also has an additional peak in longer wavelengths, namely, a first longwave peak at about 279 nm and a second longwave peak at about 297 nm, each of which has a transmittance that is about the same or greater than the shortwave peak at about 242 nm. A chart illustrating transmittance is provided in FIG. 7A. Transmittance of light having an incident angle of zero degrees is shown by the solid line.

The physical construction of layers is provided in Table 4. The substrate is an amorphous fused silica (7980 Standard) upon which the alternating layers of high index of refraction (HfO₂) and low index of refraction (SiO₂) are disposed. The filter stack has 15 layers and a total thickness of 606.3 nm.

TABLE 4
Filter stack according to Example 0.

| | | |
|---|---|---|
| | | Physical |
| Layer | Material | Thickness (nm) |

| | |
|---|---|
| Substrate | 7980 Standard |

| | | |
|---|---|---|
| 1 | HfO2 | 91.79 |
| 2 | SiO2 | 52.64 |
| 3 | HfO2 | 34.31 |
| 4 | SiO2 | 123.73 |
| 5 | HfO2 | 38.39 |
| 6 | SiO2 | 119.52 |
| 7 | HfO2 | 30.92 |
| 8 | SiO2 | 142.82 |
| 9 | HfO2 | 18.52 |
| 10 | SiO2 | 60.43 |
| 11 | HfO2 | 76.26 |
| 12 | SiO2 | 140.98 |
| 13 | HfO2 | 29.54 |
| 14 | SiO2 | 41.49 |
| 15 | HfO2 | 44.36 |
| 16 | SiO2 | 100.25 |
| 17 | HfO2 | 42.62 |
| Medium | Air | |

| | |
|---|---|
| Total Thickness | 1188.57 |

Example 4 was evaluated to determine how the optical filter stack would affect emissions of far-UV light, particularly light generated by a KrCl excimer bulb. The optical filter stack according to Example 4 is modeled to provide a peak emission at 221.9 nm for light having an incident angle of zero degrees, an emission at 240 nm having an intensity of 1.24% of the peak emission, an emission at 241 nm having an intensity of 1.30% of the peak emission, an emission at 242 nm having an intensity of 1.19% of the peak emission, an emission at 243 nm having an intensity of 1.03% of the peak emission, an emission at 250 nm having an intensity of 0.05% of the peak emission, and an emission at 258 nm having an intensity of 0.03% of the peak emission. A 1% emission relative to the peak emission at 221.9 nm is reached at 243.05 nm. FIG. 7B provides a chart illustrating the emission profile of Example 4 (black) in comparison with unfiltered emissions from a KrCl excimer bulb (gray).

### Example 5

An optical filter stack was designed that largely passes far-UV light having (1) a wavelength of about 230 nm and shorter and (2) a wavelength of about 275 and longer. The filter stack was also designed to transmit at least 3% of each wavelength from 230 nm and 283 nm as measured at an incident angle of zero degrees. The peak transmittance is modeled to occur at about 223 nm.

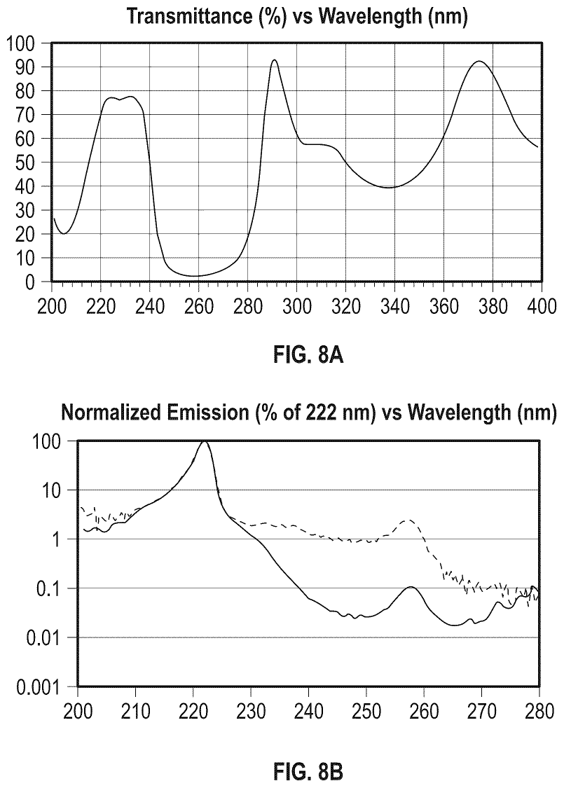

Example 0 is designed to provide about 90% of peak transmittance at about 227 nm, about 75% of peak transmittance at about 229 nm, about 50% of peak transmittance at about 231 nm, about 25% of peak transmittance at about 233 nm, about 10% of peak transmittance at about 236.5, about 5% of peak transmittance at 240 nm, and about 3% of transmittance from about 245 nm to about 255 nm, for light having an incident angle of zero degrees. The filter stack also has an additional peak in longer wavelengths, namely, longwave peaks at about 281 nm and about 365 each having a greater transmittance than the 223 nm shortwave peak transmittance. A chart illustrating transmittance is provided in FIG. 8A. Transmittance of light having an incident angle of zero degrees is shown by the solid line.

The physical construction of layers is provided in Table 5. The substrate is an amorphous fused silica (7980 Standard) upon which the alternating layers of high index of refraction (HfO₂) and low index of refraction (SiO₂) are disposed. The filter stack has 16 layers and a total thickness of 891.53 nm.

TABLE 5
Filter stack according to Example 5.

| | | |
|---|---|---|
| | | Physical |
| Layer | Material | Thickness (nm) |

| | |
|---|---|
| Substrate | 7980 Standard |

| | | |
|---|---|---|
| 1 | HfO2 | 82.77 |
| 2 | SiO2 | 51.6 |
| 3 | HfO2 | 25.56 |
| 4 | SiO2 | 127.31 |
| 5 | HfO2 | 27.82 |
| 6 | SiO2 | 48.05 |
| 7 | HfO2 | 23.69 |
| 8 | SiO2 | 45.46 |
| 9 | HfO2 | 80.15 |
| 10 | SiO2 | 47.53 |
| 11 | HfO2 | 27.02 |
| 12 | SiO2 | 53.88 |
| 13 | HfO2 | 14.95 |
| 14 | SiO2 | 73.26 |
| 15 | HfO2 | 31.84 |
| 16 | SiO2 | 130.66 |
| Medium | Air | |

| | |
|---|---|
| Total Thickness | 891.53 |

Example 5 was evaluated to determine how the optical filter stack would affect emissions of far-UV light, particularly light generated by a KrCl excimer bulb. The optical filter stack according to Example 5 is modeled to provide a peak emission at 221.9 nm for light having an incident angle of zero degrees, an emission at 230 nm having an intensity of 1.22% of the peak emission, an emission at 230 nm having an intensity of 0.99% of the peak emission, and an emission at 258 nm having an intensity of 0.11% of the peak emission. A 1% emission relative to the peak emission at 221.9 nm is reached at 230.89 nm. FIG. 8B provides a chart illustrating the emission profile of Example 5 (black) in comparison with unfiltered emissions from a KrCl excimer bulb (gray).

### Example 6

An optical filter stack was designed that largely passes far-UV light having (1) a wavelength of about 230 nm and shorter and (2) a wavelength of about 283 nm and longer. The filter stack was also designed to transmit at least 10% of each wavelength from 230 nm and 283 nm as measured at an incident angle of zero degrees. The peak transmittance is modeled to occur at about 223 nm.

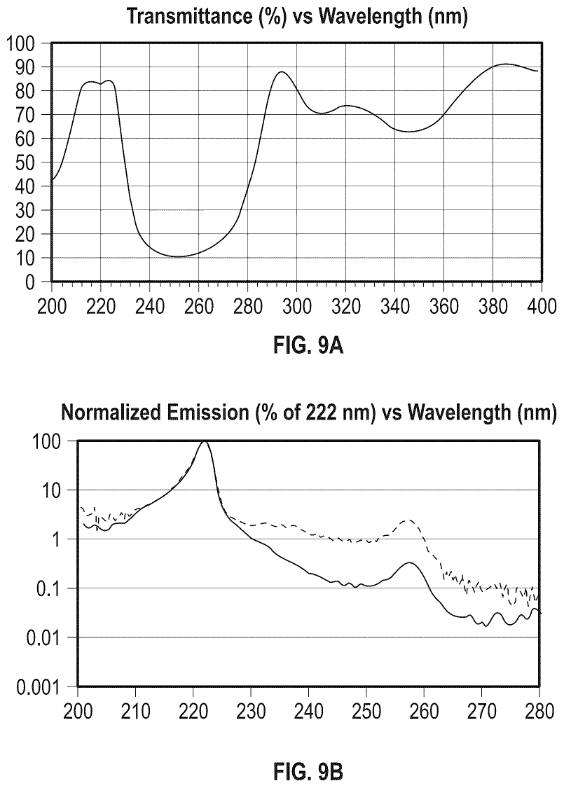

Example 6 is designed to provide about 90% of peak transmittance at about 226 nm, about 75% of peak transmittance at about 228 nm, about 50% of peak transmittance at about 230.5 nm, about 25% of peak transmittance at about 235 nm, and about 13% of peak transmittance at about 245 nm to about 255 nm, for light having an incident angle of zero degrees. The filter stack also has an additional peak in longer wavelengths, namely, a longwave peak at about 294 nm that is greater than 80% of the peak transmittance. A chart illustrating transmittance is provided in FIG. 9A. Transmittance of light having an incident angle of zero degrees is shown by the solid line.

The physical construction of layers is provided in Table 6. The substrate is an amorphous fused silica (7980 Standard) upon which the alternating layers of high index of refraction (HfO₂) and low index of refraction (SiO₂) are disposed. The filter stack has 12 layers and a total thickness of 688.91 nm.

TABLE 6
Filter stack according to Example 6.

| | | |
|---|---|---|
| | | Physical |
| Layer | Material | Thick (nm) |

| | |
|---|---|
| Substrate | 7980 Standard |

| | | |
|---|---|---|
| 1 | HfO2 | 58.31 |
| 2 | SiO2 | 136.48 |
| 3 | HfO2 | 36.19 |
| 4 | SiO2 | 121.78 |
| 5 | HfO2 | 20.11 |
| 6 | SiO2 | 41.66 |
| 7 | HfO2 | 33.74 |
| 8 | SiO2 | 50.15 |
| 9 | HfO2 | 34.53 |
| 10 | SiO2 | 29.53 |
| 11 | HfO2 | 20.11 |
| 12 | SiO2 | 106.32 |
| Medium | Air | |

| | |
|---|---|
| Total Thickness | 688.91 |

Example 6 was evaluated to determine how the optical filter stack would affect emissions of far-UV light, particularly light generated by a KrCl excimer bulb. The optical filter stack according to Example 6 is modeled to provide a peak emission at 221.9 nm for light having an incident angle of zero degrees, an emission at 229 nm having an intensity of 1.29% of the peak emission, an emission at 230 nm having an intensity of 1.07% of the peak emission, an emission at 231 nm having an intensity of 0.94% of the peak emission, an emission at 240 nm having an intensity of 0.20% of the peak emission, an emission at 250 nm having an intensity of 0.12% of the peak emission, and an emission at 258 nm having an intensity of 0.34% of the peak emission. A 1% emission relative to the peak emission at 221.9 nm is reached at 230.28 nm. FIG. 9B provides a chart illustrating the emission profile of Example 6 (black) in comparison with unfiltered emissions from a KrCl excimer bulb (gray).

### Comparative Example—Unfiltered KrCl Lamp

An unfiltered krypton chloride far-UV lamp was tested as a comparative example. FIGS. 4-9 illustrates the emissions of an unfiltered far-UV lamp (shown in gray) in comparison with far-UV lamps having exemplified optical filters. Unfiltered far-UV light emits UV radiation at wavelengths that are potentially harmful to human tissues. To avoid potential harm, any application of such unfiltered light must limit human exposure. Krypton chloride emits substantial radiation around 258 nm, which can significantly contribute to the overall risk and exposure limits of any device utilizing such unfiltered radiation.

The emissions from an unfiltered krypton chloride far-UV lamp provide a peak emission at 221.9 nm, an emission at about 240 nm having an intensity of about 1.20% of the peak emission, an emission at about 241 nm having an intensity of about 1.25% of the peak emission, an emission at about 242 nm having an intensity of about 1.12% of the peak emission, an emission at about 243 nm having an intensity of about 1.09% of the peak emission, an emission at about 250 nm having an intensity of 0.89% of the peak emission, and an emission at about 258 nm having an intensity of about 2.31% of the peak emission.

### Example 7

An optical filter stack was designed that largely passes far-UV light having (1) a wavelength of about 215 to about 242 nm, and (2) a wavelength of about 310 nm and longer. The filter stack was also designed to transmit at least a substantial amount of each wavelength from 220 nm to 320 nm as measured at an incident angle of zero degrees. Wavelengths around 250-280 had a transmittance of about 4% or greater, and at least 3.5% or greater. The peak transmittance from 222 nm to 245 nm was configured to reside at around about 230 to about 240 nm. The optical filter stack was also configured to have a second major transmittance peak around 320.

The physical construction of layers is provided in Table 7. The substrate is an amorphous fused silica (7980 Standard) from Corning Inc (Corning, NY) upon which the alternating layers of high index of refraction (HfO₂) and low index of refraction (SiO₂) were disposed. The filter stack has 13 layers and a total thickness of 636.62 nm.

TABLE 7
Filter stack according to Example 7.

| | | |
|---|---|---|
| | | Physical |
| Layer | Material | Thick (nm) |

| | |
|---|---|
| Substrate | 7980 Standard |

| | | |
|---|---|---|
| 1 | HfO2 | 82.7 |
| 2 | SiO2 | 65.6 |
| 3 | HfO2 | 22.5 |
| 4 | SiO2 | 62.6 |
| 5 | HfO2 | 22.3 |
| 6 | SiO2 | 52.6 |
| 7 | HfO2 | 21.9 |
| 8 | SiO2 | 59.6 |
| 9 | HfO2 | 23.6 |
| 10 | SiO2 | 59.4 |
| 11 | HfO2 | 18.3 |
| 12 | SiO2 | 48.0 |
| 13 | HfO2 | 43.3 |
| Medium | Air | |

| | |
|---|---|
| Total Thickness | 636.62 |

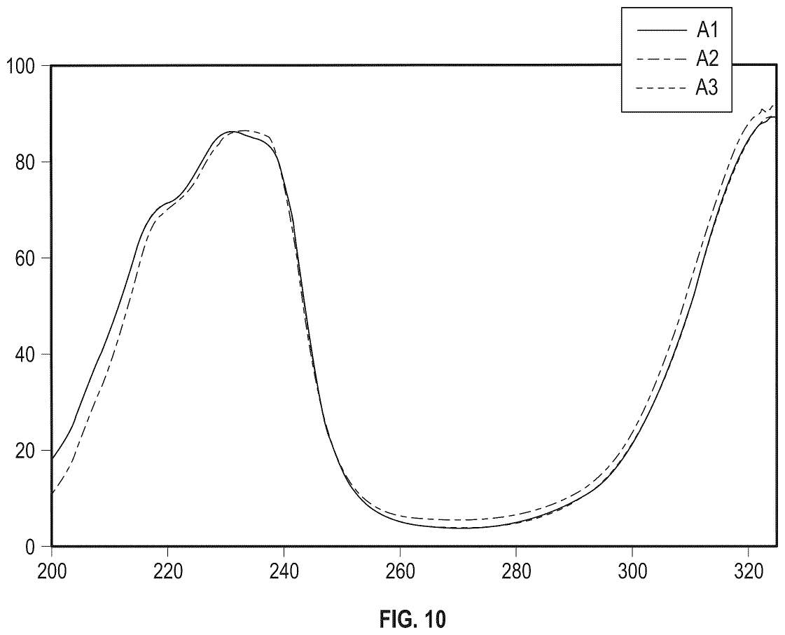

Three optical filter stacks (Stack A1, Stack A2, and Stack A3) were manufactured based on the design. The empirical performance of each of the three optical filter stacks closely matched the target filter curve design as illustrated in FIG. 10. All results performed as desired. As shown in FIG. 10, each of the manufactured optical filter stacks exhibited a local peak transmittance around about 230 to about nm 240 nm, and a 50% cutoff that is substantially above 240 nm (the 50% transmittance point on slope between the local band pass peak around about 230 nm to about 240 nm and the band stop region around about 260 nm to about 280 nm). Each of the manufactured optical filter stacks also showed significant transmittance above 310 nm, and second 50% cutoff that is substantially above 300 nm. Each filter stack also attenuated transmittance below 210 nm, which provides a pass around the 200 nm or lower wavelengths that can be responsible for undesirable ozone generation.

### Example 8

An optical filter stack was designed that largely passes far-UV light having (1) a wavelength of about 215 to about 235 nm, and (2) a wavelength of about 290 nm and longer. The filter stack was also designed to transmit at least a substantial amount of a wavelength above 240 as measured at an incident angle of zero degrees. The peak transmittance from 222 nm to 245 nm was configured to reside at around about 220 to about 235 nm. The optical filter stack was also configured to have one or more additional major transmittance peaks around above 280 nm.

The physical construction of layers is provided in Table 8. The substrate is an amorphous fused silica (7980 Standard) from Corning Inc (Corning, NY), Heraeus SUPRASIL synthetic fused silica from Heraeus (Hanau, Germany), and JGS1 glass, upon which the alternating layers of high index of refraction (HfO₂) and low index of refraction (SiO₂) were disposed. This set of examples includes some examples that included various annealing steps after coating: annealing conditions utilized in these examples include no annealing, annealing for 200° C. for 4 h after coating, or annealing at 400° C. for 4 h after coating. Annealing was tested to explore improved adhesion of layers to each other and to the substrate. The filter stack has 13 layers and a total thickness of 953.71 nm.

TABLE 8
Filter stack according to Example 0.

| | | |
|---|---|---|
| | | Physical |
| Layer | Material | Thick (nm) |

| | |
|---|---|
| Substrate | Various fused silica |

| | | |
|---|---|---|
| 1 | HfO2 | 34.08 |
| 2 | SiO2 | 129.43 |
| 3 | HfO2 | 50.2 |
| 4 | SiO2 | 15.27 |
| 5 | HfO2 | 56.89 |
| 6 | SiO2 | 131.14 |
| 7 | HfO2 | 28.55 |
| 8 | SiO2 | 45 |
| 9 | HfO2 | 85.59 |
| 10 | SiO2 | 41.99 |
| 11 | HfO2 | 29.25 |
| 12 | SiO2 | 42.58 |
| 13 | HfO2 | 29.12 |
| 14 | SiO2 | 43.81 |
| 15 | HfO2 | 31.7 |
| 16 | SiO2 | 43.65 |
| 17 | HfO2 | 31.55 |
| 18 | SiO2 | 83.91 |
| Medium | Air | |

| | |
|---|---|
| Total Thickness | 953.71 |

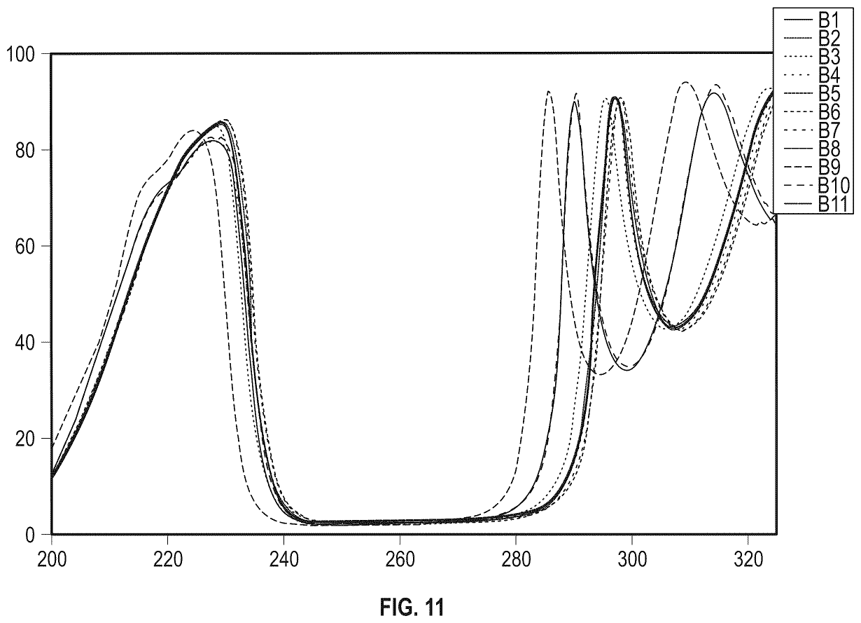

Eleven optical filter stacks (Stack B1, Stack B2, Stack B3, Stack B4, Stack B5, Stack B6, Stack B7, Stack B8, Stack B9, Stack B10, and Stack B11) were manufactured based on the design. The empirical performance of each of the eleven optical filter stacks closely matched the target filter curve design as illustrated in FIG. 11, although certain manufactured optical filter stacks were frequency shifted relative to others, for example, Stack B9 has a peak near about 222 nm and about 280 nm, whereas B3 has a peak near about 235 nm and about 295 nm. All results performed as desired. As shown in FIG. 11, each of the manufactured optical filter stacks exhibited a local peak transmittance around about 222 to about nm 235 nm, and a 50% cutoff that is substantially above 230 nm (the 50% transmittance point on slope between the local band pass peak around about 230 nm to about 240 nm and the band stop region around about 260 nm to about 280 nm). Each of the manufactured optical filter stacks also showed significant transmittance above 280 nm.

### Example 9—Visible Light Reflective Optical Filter Stack

An optical filter stack was designed that significantly (1) passes far-UV light having a wavelength of about 215 nm to about 235 nm, and (2) reflects one or more wavelength of about 440 nm to about 750 nm. The filter stack was also designed to have low reflectance at one or more wavelength between 380 and 440.

This example of an optical filter stack utilizes additional intermediate layers that are neither the high index of refraction layer or the low index of refraction layer, and these intermediate layers can be placed between the high index of refraction layers (HfO₂) and the low index of refraction layers (SiO₂), or in place of one of the high index of refraction layers (HfO₂). This example also illustrates how the layers of a filter stack can be disposed on both sides of a substrate, yet still be configured so that taken together they provide the target transmittance curve. This example yet further illustrates how an optical filter stack can be designed to provide a target reflectance curve while also being designed to achieve a target transmittance curve.

The physical construction of layers is provided in Table 9. The substrate is an amorphous fused silica (7980 Standard) upon which layers of high index of refraction (HfO₂), intermediate layers suitable for reflecting visible light, and layers of low index of refraction (SiO₂) are disposed. The optical filter stack includes 13 layers on a first side with a thickness of 1565.61 and 12 layers on a second side with a thickness of 1263.11, so as to result in 25 layers total upon the substrate.

TABLE 9A
Filter stack according to Example 9, Side 1
Side 1

| | | |
|---|---|---|
| | | Physical |
| Layer | Material | Thick (nm) |

| | |
|---|---|
| Substrate | 7980 Standard |

| | | |
|---|---|---|
| 1 | HfO2 | 27.25 |
| 2 | SiO2 | 220.91 |
| 3 | Intermediate Layer | 7.46 |
| 4 | SiO2 | 205.68 |
| 5 | Intermediate Layer | 7.14 |
| 6 | SiO2 | 211.98 |
| 7 | Intermediate Layer | 7.38 |
| 8 | SiO2 | 428.01 |
| 9 | Intermediate Layer | 11.35 |
| 10 | HfO2 | 46.29 |
| 11 | SiO2 | 278.54 |
| 12 | Intermediate Layer | 12.21 |
| 13 | SiO2 | 101.4 |
| Medium | Air | |

| | |
|---|---|
| Total Thickness | 1565.61 |

TABLE 9B
Filter stack according to Example 9, Side 2
Side 2

| Layer | Material | Physical |
|---|---|---|
| Substrate | 7980 Standard | Thick (nm) |
| 1 | HfO2 | 23.78 |
| 2 | SiO2 | 215.79 |
| 3 | Intermediate Layer | 13.38 |
| 4 | SiO2 | 349.33 |
| 5 | Intermediate Layer | 17.37 |
| 6 | SiO2 | 201.81 |
| 7 | Intermediate Layer | 10 |
| 8 | SiO2 | 281.27 |
| 9 | HfO2 | 19.77 |
| 10 | SiO2 | 54.79 |
| 11 | Intermediate Layer | 10 |
| 12 | SiO2 | 65.8 |
| Medium | Air | |

| | |
|---|---|
| Total Thickness | 1263.11 |

Additionally, either side alone can be used to provide a filter stack that predominantly passes far-UV light having a wavelength of about 215 nm to about 235 nm, and (2) reflects one or more wavelength of about 440 nm to about 750 nm. However, the combined example provided additional control for achieving a transmittance that focuses on passing 222 nm while having high reflectance of non-purple visible light. The effect of providing high reflectance in the visible light region is to obscure the purple-type appearance that can arise in far-UV sources like bulbs, emission tube, and excimer lamps.

Such optical filter stacks can be described in terms of their transmittance and their reflectance. The optical filter stack had a peak transmittance between about 215 nm and about 230 nm. The optical filter stack included multiple reflectance peaks, and included a region of high reflectance between about 420 nm and about 580 nm. The optical filter stack had a peak reflectance between had a peak transmittance at about 540 nm to about 550 nm when considering the region between 420 nm and 580 nm. A reflectance peak in the 500 nm to 750 nm range provides non-purple hues that are useful for offsetting unusual coloring generated by far-UV radiation sources. The described reflective optical filter stack is one example of a reflective optical filter stack that can be useful for reflecting room light back into the room in a manner that obscures the purple-pink appearance that can occur with far-UV radiation sources, and can make a far-UV radiation source appear more like a conventional light source.

### EXEMPLIFIED ASPECTS

The following aspects are provided as example aspects of the various disclosed subject matter:
Aspect 1 provides an optical filter stack having layers of a high refractive index dielectric material and layers a low refractive index dielectric material each having an optical thickness configured to together provide a transmittance curve with a transmittance of at least 70% at 222 nm and substantial transmittance of one or more wavelength from 240 nm to 280 nm, as measured at an incident angle of zero degrees.
Aspect 2 provides an optical filter stack having layers of a high refractive index dielectric material and layers a low refractive index dielectric material each having an optical thickness configured to together provide a transmittance curve with a transmittance of at least 70% at 222 nm, one or more wavelength from 230 nm to 245 nm having a transmittance about the same or greater than the transmittance at 222 nm, and each wavelength from 255 nm to 265 nm has a transmittance of less than 15% of the transmittance at 222 nm, as measured at an incident angle of zero degrees.
Aspect 3 provides an optical filter stack having layers of a high refractive index dielectric material and layers a low refractive index dielectric material each having an optical thickness configured to together provide a transmittance curve with a transmittance of at least 70% at 222 nm, a transmittance at one or more wavelengths from 240 nm to 245 nm that is about 85% or more of a peak transmittance from 222 nm to 245 nm, and a transmittance at one or more wavelengths from 255 nm to 265 nm that is about 15% or less of the peak transmittance from 222 nm to 245 nm, as measured at an incident angle of zero degree.
Aspect 4 provides an optical filter stack having layers of a high refractive index dielectric material and layers a low refractive index dielectric material each having an optical thickness configured to together provide a transmittance curve with a transmittance of at least 70% at 222 nm, a transmittance at one or more wavelength from 242 nm to 252 that is 50% of a peak transmittance from 222 nm to 242 nm, and a transmittance at one or more wavelengths from 255 nm to 265 nm that is at least 3% of the peak transmittance from 222 nm to 245 nm, as measured at an incident angle of zero degrees.
Aspect 5 provides an optical filter stack having layers of a high refractive index dielectric material and layers a low refractive index dielectric material each having an optical thickness configured to together provide a transmittance curve with a transmittance of at least 70% at 222 nm, a transmittance at one or more wavelengths from 255 nm to 265 nm that is 3% to 15% of a peak transmittance from 222 nm to 245 nm, and a transmittance at one or more wavelengths from 270 nm to 280 nm that is 20% or greater than the peak transmittance from 222 nm to 245 nm, as measured at an incident angle of zero degrees.
Aspect 6 provides an optical filter stack having layers of a high refractive index dielectric material and layers a low refractive index dielectric material each having an optical thickness configured to together provide a transmittance curve with a transmittance of at least 70% at 222 nm, a transmittance at one or more wavelengths from 255 nm to 265 nm that is 3% to 15% of a peak transmittance from 222 nm to 245 nm, and a transmittance at one or more wavelengths from 270 nm to 370 that is 90% or greater than the peak transmittance from 222 nm to 245 nm, as measured at an incident angle of zero degrees.
Aspect 7 provides an optical filter stack having layers of a high refractive index dielectric material and layers a low refractive index dielectric material each having an optical thickness configured to together provide a transmittance curve with a transmittance of at least 70% at 222 nm, a transmittance at one or more wavelength from 230 nm to 240 that is 50% of a peak transmittance from 222 nm to 230 nm, a transmittance at one or more wavelengths from 245 nm to 260 nm that is 3% to 15% of the peak transmittance from 222 nm to 230 nm, a transmittance at one or more wavelengths from 270 nm to 280 nm that is 20% or greater than the peak transmittance from 222 nm to 230 nm, and a transmittance at one or more wavelengths from 270 nm to 370 nm that is 90% or greater than the peak transmittance from 222 nm to 230 nm, as measured at an incident angle of zero degrees.
Aspect 8 provides the optical filter stack of any one of Aspects 1 to 7, wherein one or more wavelength from 230 nm to 370 nm has a transmittance about the same or greater than the transmittance at 222 nm, as measured at an incident angle of zero degrees.
Aspect 9 provides the optical filter stack of any one of Aspects 1 to 8, wherein one or more wavelength from 237 nm to 370 nm has a transmittance about the same or greater than the transmittance at 222 nm, as measured at an incident angle of zero degrees.
Aspect 10 provides the optical filter stack of any one of Aspects 1 to 9, wherein one or more wavelength from 240 nm to 370 nm has a transmittance about the same or greater than the transmittance at 222 nm, as measured at an incident angle of zero degrees.
Aspect 11 provides the optical filter stack of any one of Aspects 1 to 10, wherein one or more wavelength from 230 nm to 250 nm has a transmittance about the same or greater than the transmittance at 222 nm, as measured at an incident angle of zero degrees.
Aspect 12 provides the optical filter stack of any one of Aspects 1 to 11, wherein one or more wavelength from 237 nm to 250 nm has a transmittance about the same or greater than the transmittance at 222 nm, as measured at an incident angle of zero degrees.
Aspect 13 provides the optical filter stack of any one of Aspects 1 to 12, wherein one or more wavelength from 240 nm to 250 nm has a transmittance about the same or greater than the transmittance at 222 nm, as measured at an incident angle of zero degrees.
Aspect 14 provides the optical filter stack of any one of Aspects 1 to 13, wherein each wavelength from 230 nm to 240 nm has a transmittance at least 70%, as measured at an incident angle of zero degrees.
Aspect 15 provides the optical filter stack of any one of Aspects 1 to 14, wherein each wavelength from 230 nm to 240 nm has a transmittance at least 90% of the transmittance at 222 nm, as measured at an incident angle of zero degrees.
Aspect 16 provides the optical filter stack of any one of Aspects 1 to 15, wherein each wavelength from 230 nm to 240 nm has a transmittance at least 85% of a peak transmittance from 222 nm to 245 nm, as measured at an incident angle of zero degrees.
Aspect 17 provides the optical filter stack of any one of Aspects 1 to 16, wherein each wavelength from 230 nm to 241 nm has a transmittance at least 60%, as measured at an incident angle of zero degrees.
Aspect 18 provides the optical filter stack of any one of Aspects 1 to 17, wherein each wavelength from 230 nm to 241 nm has a transmittance at least 80% of the transmittance at 222 nm, as measured at an incident angle of zero degrees.
Aspect 1 provides the optical filter stack of any one of Aspects 1 to 18, wherein each wavelength from 230 nm to 241 nm has a transmittance at least 75% of a peak transmittance from 222 nm to 245 nm, as measured at an incident angle of zero degrees.
Aspect 20 provides the optical filter stack of any one of Aspects 1 to 19, wherein each wavelength from 230 nm to 242 nm has a transmittance at least 50%, as measured at an incident angle of zero degrees.
Aspect 21 provides the optical filter stack of any one of Aspects 1 to 20, wherein each wavelength from 230 nm to 242 nm has a transmittance at least 60% of the transmittance at 222 nm, as measured at an incident angle of zero degrees.
Aspect 22 provides the optical filter stack of any one of Aspects 1 to 21, wherein each wavelength from 230 nm to 242 nm has a transmittance at least 55% of a peak transmittance from 222 nm to 245 nm, as measured at an incident angle of zero degrees.
Aspect 23 provides the optical filter stack of any one of Aspects 1 to 22, wherein each wavelength from 230 nm to 245 nm has a transmittance at least 20%, as measured at an incident angle of zero degrees.
Aspect 24 provides the optical filter stack of any one of Aspects 1 to 23, wherein each wavelength from 230 nm to 245 nm has a transmittance at least 25% of the transmittance at 222 nm, as measured at an incident angle of zero degrees.
Aspect 25 provides the optical filter stack of any one of Aspects 1 to 24, wherein each wavelength from 230 nm to 245 nm has a transmittance at least 23% of a peak transmittance from 222 nm to 245 nm, as measured at an incident angle of zero degrees.
Aspect 26 provides the optical filter stack of any one of Aspects 1 to 25, wherein each wavelength from 230 nm to 248 nm has a transmittance at least 10%, as measured at an incident angle of zero degrees.
Aspect 27 provides the optical filter stack of any one of Aspects 1 to 26, wherein each wavelength from 230 nm to 248 nm has a transmittance at least 10% of the transmittance at 222 nm, as measured at an incident angle of zero degrees.
Aspect 28 provides the optical filter stack of any one of Aspects 1 to 27, wherein each wavelength from 230 nm to 248 nm has a transmittance at least 10% of a peak transmittance from 222 nm to 245 nm, as measured at an incident angle of zero degrees.
Aspect 29 provides the optical filter stack of any one of Aspects 1 to 28, wherein each wavelength from 230 nm to 250 nm has a transmittance at least 5%, as measured at an incident angle of zero degrees.
Aspect 30 provides the optical filter stack of any one of Aspects 1 to 29, wherein each wavelength from 230 nm to 250 nm has a transmittance at least 5% of the transmittance at 222 nm, as measured at an incident angle of zero degrees.
Aspect 31 provides the optical filter stack of any one of Aspects 1 to 1, wherein each wavelength from 230 nm to 250 nm has a transmittance at least 5% of a peak transmittance from 222 nm to 245 nm, as measured at an incident angle of zero degrees.
Aspect 32 provides the optical filter stack of any one of Aspects 1 to 31, wherein each wavelength from 230 nm to 255 nm has a transmittance at least 3%, as measured at an incident angle of zero degrees.
Aspect 33 provides the optical filter stack of any one of Aspects 1 to 32, wherein each wavelength from 230 nm to 255 nm has a transmittance at least 3% of the transmittance at 222 nm, as measured at an incident angle of zero degrees.
Aspect 34 provides the optical filter stack of any one of Aspects 1 to 33, wherein each wavelength from 230 nm to 255 nm has a transmittance at least 3% of a peak transmittance from 222 nm to 245 nm, as measured at an incident angle of zero degrees.
Aspect 35 provides the optical filter stack of any one of Aspects 1 to 34, wherein each wavelength from 230 nm to 260 nm has a transmittance at least 3%, as measured at an incident angle of zero degrees.
Aspect 2 provides the optical filter stack of any one of Aspects 1 to 35, wherein each wavelength from 230 nm to 260 nm has a transmittance at least 3% of the transmittance at 222 nm, as measured at an incident angle of zero degrees.
Aspect 37 provides the optical filter stack of any one of Aspects 1 to 36, wherein each wavelength from 230 nm to 260 nm has a transmittance at least 3% of a peak transmittance from 222 nm to 245 nm, as measured at an incident angle of zero degrees.
Aspect 38 provides the optical filter stack of any one of Aspects 1 to 37, wherein each wavelength from 255 nm to 265 nm has a transmittance of less than 15%, as measured at an incident angle of zero degrees.
Aspect 39 provides the optical filter stack of any one of Aspects 1 to 38, wherein each wavelength from 255 nm to 265 nm has a transmittance of less than 15% of the transmittance at 222 nm, as measured at an incident angle of zero degrees.
Aspect 40 provides the optical filter stack of any one of Aspects 1 to 39, wherein each wavelength from 255 nm to 265 nm has a transmittance of less than 15% of a peak transmittance from 222 nm to 245 nm, as measured at an incident angle of zero degrees.
Aspect 41 provides the optical filter stack of any one of Aspects 1 to 40, wherein a wavelength at 258 nm has a transmittance that is about 4% to about 15%, as measured at an incident angle of zero degrees.
Aspect 42 provides the optical filter stack of any one of Aspects 1 to 41, wherein a wavelength at 258 nm has a transmittance that is about 4% to about 15% of the transmittance at 222 nm, as measured at an incident angle of zero degrees.
Aspect 43 provides the optical filter stack of any one of Aspects 1 to 42, wherein a wavelength at 258 nm has a transmittance that is about 4% to about 15% of a peak transmittance from 222 nm to 245 nm, as measured at an incident angle of zero degrees.
Aspect 44 provides the optical filter stack of any one of Aspects 1 to 43, wherein one or more wavelength from 270 nm to 370 nm has a transmittance at least 90% of the transmittance at 222 nm, as measured at an incident angle of zero degrees.
Aspect 45 provides the optical filter stack of any one of Aspects 1 to 44, wherein one or more wavelength from 270 nm to 370 nm has a transmittance about the same or greater than the transmittance at 222 nm, as measured at an incident angle of zero degrees.
Aspect 46 provides the optical filter stack of any one of Aspects 1 to 45, wherein one or more wavelength from 270 nm to 370 nm has a transmittance at least 90% of a peak transmittance from 222 nm to 245 nm, as measured at an incident angle of zero degrees.
Aspect 47 provides the optical filter stack of any one of Aspects 1 to 46, wherein one or more wavelength from 242 nm to 252 nm has a transmittance that is 50% of a peak transmittance from 222 nm to 242.
Aspect 48 provides the optical filter stack of any one of Aspects 1 to 47, which provides a transmittance of at least 70% at 222 nm, when measured at an incident angle of 0-25 degrees.
Aspect 49 provides the optical filter stack of any one of Aspects 1 to 48, which has a greater transmittance at 222 nm as measured at an incident angle of 10 degrees than the transmittance at 222 nm as measured at an incident angle of zero degrees.
Aspect 50 provides the optical filter stack of any one of Aspects 1 to 49, which has a lower transmittance at one or more wavelength shorter than 210 nm as measured at an incident angle of 10 degrees compared to a transmittance of the wavelength shorter than 210 as measured at an incident angle of zero degrees.
Aspect 51 provides the optical filter stack of any one of Aspects 1 to 50, which has a lower transmittance at 258 nm as measured at an incident angle of 10 degrees compared to a transmittance at 258 nm as measured at an incident angle of zero degrees.
Aspect 52 provides the optical filter stack of any one of Aspects 1 to 51, which one or more wavelength from 237 nm to 240 nm has a peak transmittance about the same or greater than the transmittance at 222 nm; one or more wavelength from 241 nm to 244 nm has a transmittance that is about 50% of the peak transmittance; one or more wavelength from 244 nm to 247 nm has a transmittance that is about 20% of the peak transmittance; and a 258 nm wavelength has a transmittance of 2% to 6% of the peak transmittance.
Aspect 53 provides the optical filter stack of any one of Aspects 1 to 51, wherein one or more wavelength from 235 nm to 239 nm has a peak transmittance about the same or greater than the transmittance at 222 nm; one or more wavelength from 242 nm to 245 nm has a transmittance that is about 50% of the peak transmittance; one or more wavelength from 249 nm to 252 nm has a transmittance that is about 20% of the peak transmittance; and a 258 nm wavelength has a transmittance of 10% to 15% of the peak transmittance.
Aspect 54 provides the optical filter stack of any one of Aspects 1 to 51, wherein one or more wavelength from 240 nm to 244 nm has a peak transmittance about the same or greater than the transmittance at 222 nm; one or more wavelength from 247 nm to 250 nm has a transmittance that is about 50% of the peak transmittance; one or more wavelength from 251 nm to 254 nm has a transmittance that is about 20% of the peak transmittance; and a 258 nm wavelength has a transmittance of 3% to 10% of the peak transmittance.
Aspect 55 provides the optical filter stack of any one of Aspects 1 to 51, wherein one or more wavelength from 240 nm to 243 nm has a peak transmittance about the same or greater than the transmittance at 222 nm; one or more wavelength from 244 nm to 246 nm has a transmittance that is about 50% of the peak transmittance; one or more wavelength from 246 nm to 248 nm has a transmittance that is about 20% of the peak transmittance; and a 258 nm wavelength has a transmittance of 0.1% to 5% of the peak transmittance.
Aspect 56 provides the optical filter stack of any one of Aspects 1 to 51, wherein one or more wavelength from 230 nm to 232 nm has a transmittance that is about 50% of the peak transmittance; one or more wavelength from 233 nm to 235 nm has a transmittance that is about 20% of the peak transmittance; a 258 nm wavelength has a transmittance of 3% to 10% of the peak transmittance; and one or more wavelength from 270 nm to 280 nm has a transmittance that is about 30% or more of the peak transmittance.
Aspect 57 provides the optical filter stack of any one of Aspects 1 to 51, wherein one or more wavelength from 229 nm to 232 nm has a transmittance that is about 50% of the peak transmittance; one or more wavelength from 233 nm to 240 nm has a transmittance that is about 20% of the peak transmittance; a 258 nm wavelength has a transmittance of 10% to 15% of the peak transmittance; and one or more wavelength from 270 nm to 280 nm has a transmittance that is about 30% or more of the peak transmittance.
Aspect 58 provides the optical filter stack of any one of Aspects 1 to 57, which has about 7 to about 30 layers.
Aspect 59 provides the optical filter stack of any one of Aspects 1 to 58, which has about 10 to 20 layers.
Aspect 60 provides the optical filter stack of any one of Aspects 1 to 59, which has 16 or fewer layers.
Aspect 61 provides the optical filter stack of any one of Aspects 1 to 60, wherein the layers taken together have a thickness of about 2 micrometer or less.
Aspect 62 provides the optical filter stack of any one of Aspects 1 to 61, wherein the layers taken together have a thickness of 500 nm to 2,000 nm.
Aspect 3 provides the optical filter stack of any one of Aspects 1 to 62, wherein the layers taken together have a thickness of 600 nm to 1,300 nm.
Aspect 64 provides the optical filter stack of any one of Aspects 1 to 63, wherein the low index dielectric material is SiO2.
Aspect 65 provides the optical filter stack of any one of Aspects 1 to 64, wherein the high index dielectric material is HfO2.
Aspect 4 provides the optical filter stack of any one of Aspects 1 to 65, which comprises one or more of the following layers, optionally in the following order within the filter stack starting from the first layer:
a HfO2 layer having a thickness of from 76.25 nm to 84.28 nm;
a SiO2 layer having a thickness of from 65.34 nm to 72.21 nm;
a HfO2 layer having a thickness of from 17.10 nm to 18.91 nm;
a SiO2 layer having a thickness of from 130.9 nm to 144.6 nm;
a HfO2 layer having a thickness of from 16.72 nm to 18.48 nm;
a SiO2 layer having a thickness of from 71.28 nm to 78.79 nm;
a HfO2 layer having a thickness of from 14.40 nm to 15.91 nm;
a SiO2 layer having a thickness of from 62.33 nm to 68.90 nm;
a HfO2 layer having a thickness of from 66.24 nm to 73.21 nm;
a SiO2 layer having a thickness of from 60.50 nm to 66.87 nm;
a HfO2 layer having a thickness of from 17.11 nm to 18.92 nm;
a SiO2 layer having a thickness of from 67.18 nm to 74.25 nm;
a HfO2 layer having a thickness of from 16.17 nm to 17.88 nm;
a SiO2 layer having a thickness of from 134.5 nm to 148.7 nm;
a HfO2 layer having a thickness of from 19.82 nm to 21.91 nm; and
a SiO2 layer having a thickness of from 94.23 nm to 104.1 nm.
Aspect 67 provides the optical filter stack of any one of Aspects 1 to 66, which comprises one or more of the following layers, optionally in the following order within the filter stack starting from the first layer:
a HfO2 layer having a thickness of from 78.88 nm to 87.19 nm;
a SiO2 layer having a thickness of from 51.10 nm to 56.47 nm;
a HfO2 layer having a thickness of from 34.89 nm to 38.56 nm;
a SiO2 layer having a thickness of from 34.37 nm to 37.98 nm;
a HfO2 layer having a thickness of from 47.98 nm to 53.03 nm;
a SiO2 layer having a thickness of from 82.19 nm to 90.84 nm;
a HfO2 layer having a thickness of from 30.96 nm to 34.21 nm;
a SiO2 layer having a thickness of from 48.34 nm to 53.43 nm;
a HfO2 layer having a thickness of from 32.18 nm to 35.57 nm;
a SiO2 layer having a thickness of from 43.88 nm to 48.49 nm;
a HfO2 layer having a thickness of from 37.81 nm to 41.79 nm; and
a SiO2 layer having a thickness of from 70.60 nm to 78.03 nm.
Aspect 68 provides the optical filter stack of any one of Aspects 1 to 67, which comprises one or more of the following layers, optionally in the following order within the filter stack starting from the first layer:
a HfO2 layer having a thickness of from 34.10 nm to 37.69 nm;
a SiO2 layer having a thickness of from 48.61 nm to 53.72 nm;
a HfO2 layer having a thickness of from 34.10 nm to 37.69 nm;
a SiO2 layer having a thickness of from 41.94 nm to 46.35 nm;
a HfO2 layer having a thickness of from 29.01 nm to 32.06 nm;
a SiO2 layer having a thickness of from 47.35 nm to 52.34 nm;
a HfO2 layer having a thickness of from 27.92 nm to 30.85 nm;
a SiO2 layer having a thickness of from 47.20 nm to 52.17 nm;
a HfO2 layer having a thickness of from 29.63 nm to 32.74 nm;
a SiO2 layer having a thickness of from 45.57 nm to 50.36 nm;
a HfO2 layer having a thickness of from 31.93 nm to 35.30 nm;
a SiO2 layer having a thickness of from 42.81 nm to 47.32 nm;
a HfO2 layer having a thickness of from 32.83 nm to 36.28 nm;
a SiO2 layer having a thickness of from 44.19 nm to 48.84 nm; and
a HfO2 layer having a thickness of from 38.75 nm to 42.82 nm.
Aspect 69 provides the optical filter stack of any one of Aspects 1 to 68, which comprises one or more of the following layers, optionally in the following order within the filter stack starting from the first layer:
a HfO2 layer having a thickness of from 87.20 nm to 96.37 nm;
a SiO2 layer having a thickness of from 50.00 nm to 55.27 nm;
a HfO2 layer having a thickness of from 32.59 nm to 36.02 nm;
a SiO2 layer having a thickness of from 117.5 nm to 129.9 nm;
a HfO2 layer having a thickness of from 36.47 nm to 40.30 nm;
a SiO2 layer having a thickness of from 113.5 nm to 125.4 nm;
a HfO2 layer having a thickness of from 29.37 nm to 32.46 nm;
a SiO2 layer having a thickness of from 135.6 nm to 149.9 nm;
a HfO2 layer having a thickness of from 17.59 nm to 19.44 nm;
a SiO2 layer having a thickness of from 57.40 nm to 63.45 nm;
a HfO2 layer having a thickness of from 72.44 nm to 80.07 nm;
a SiO2 layer having a thickness of from 133.9 nm to 148.0 nm;
a HfO2 layer having a thickness of from 28.06 nm to 31.01 nm;
a SiO2 layer having a thickness of from 39.41 nm to 43.56 nm;
a HfO2 layer having a thickness of from 42.14 nm to 46.57 nm;
a SiO2 layer having a thickness of from 95.23 nm to 105.2 nm; and
a HfO2 layer having a thickness of from 40.48 nm to 44.75 nm.
Aspect 70 provides the optical filter stack of any one of Aspects 1 to 69, which comprises one or more of the following layers, optionally in the following order within the filter stack starting from the first layer:
a HfO2 layer having a thickness of from 78.63 nm to 86.90 nm;
a SiO2 layer having a thickness of from 49.02 nm to 54.18 nm;
a HfO2 layer having a thickness of from 24.28 nm to 26.83 nm;
a SiO2 layer having a thickness of from 120.9 nm to 133.6 nm;
a HfO2 layer having a thickness of from 26.42 nm to 29.21 nm;
a SiO2 layer having a thickness of from 45.64 nm to 50.45 nm;
a HfO2 layer having a thickness of from 22.50 nm to 24.87 nm;
a SiO2 layer having a thickness of from 43.18 nm to 47.73 nm;
a HfO2 layer having a thickness of from 76.14 nm to 84.15 nm;
a SiO2 layer having a thickness of from 45.15 nm to 49.90 nm;
a HfO2 layer having a thickness of from 25.66 nm to 28.37 nm;
a SiO2 layer having a thickness of from 51.18 nm to 56.57 nm;
a HfO2 layer having a thickness of from 14.20 nm to 15.69 nm;
a SiO2 layer having a thickness of from 69.59 nm to 76.92 nm;
a HfO2 layer having a thickness of from 30.24 nm to 33.43 nm; and
a SiO2 layer having a thickness of from 124.1 nm to 137.1 nm.
Aspect 5 provides the optical filter stack of any one of Aspects 1 to 70, which comprises one or more of the following layers, optionally in the following order within the filter stack starting from the first layer:
a HfO2 layer having a thickness of from 55.39 nm to 61.22 nm;
a SiO2 layer having a thickness of from 129.6 nm to 143.3 nm;
a HfO2 layer having a thickness of from 34.38 nm to 37.99 nm;
a SiO2 layer having a thickness of from 115.6 nm to 127.8 nm;
a HfO2 layer having a thickness of from 19.10 nm to 21.11 nm;
a SiO2 layer having a thickness of from 39.57 nm to 43.74 nm;
a HfO2 layer having a thickness of from 32.05 nm to 35.42 nm;
a SiO2 layer having a thickness of from 47.64 nm to 52.65 nm;
a HfO2 layer having a thickness of from 32.80 nm to 36.25 nm;
a SiO2 layer having a thickness of from 28.05 nm to 31.00 nm;
a HfO2 layer having a thickness of from 19.10 nm to 21.11 nm; and
a SiO2 layer having a thickness of from 101.0 nm to 111.6 nm.
Aspect 72 provides the optical filter stack of any one of Aspects 1 to 71, which is prepared by ion-assisted deposition (IAD) electron-beam or thermal evaporation, sputtering, or plasma ion assisted deposition (PIAD).
Aspect 73 provides the optical filter stack of any one of Aspects 1 to 72, which is prepared by plasma ion-assisted deposition (PIAD).
Aspect 74 provides an optical filter stack comprising the optical filter stack of any one of Aspects 1-73 disposed on a transparent substrate.
Aspect 75 provides an optical filter stack of Aspect 74, wherein the transparent substrate is non-crystalline fused silica.
Aspect 76 provides an optical filter stack having layers of a high refractive index dielectric material and layers a low refractive index dielectric material each having an optical thickness configured to together provide a transmittance curve having a peak transmittance at a wavelength between about 215 nm and 235 nm, and a reflectance curve having a reflectance of at least 50% for one or more wavelength of visible light having a wavelength greater than 450.
Aspect 77 provides an optical filter stack of any one of Aspects 1-76, which is configured to reflect visible light.
Aspect 78 provides an optical filter stack of any one of Aspects 1-77, which is configured to reflect non-purple visible light.
Aspect 79 provides an optical filter stack of any one of Aspects 1-78, which is configured for a reflectance curve having a reflectance of at least 50% at one or more wavelength from about 430 nm to about 750 nm.
Aspect 76 provides an optical filter stack of any one of Aspects 1-75, which is configured for a reflectance curve having a reflectance of at least 60% at one or more wavelength from about 430 nm to about 750 nm.
Aspect 76 provides an optical filter stack of any one of Aspects 1-75, which is configured for a reflectance curve having a reflectance of at least 70% at one or more wavelength from about 430 nm to about 750 nm.
Aspect 76 provides an optical filter stack of any one of Aspects 1-75, which is configured for a reflectance curve having a reflectance of at least 80% at one or more wavelength from about 430 nm to about 750 nm.
Aspect 6 provides an optical filter stack of any one of Aspects 1-75, which is configured for a reflectance curve having a reflectance of at least 50% for each wavelength from about 430 nm to about 580 nm.
Aspect 76 provides an optical filter stack of any one of Aspects 1-75, which is configured for a reflectance curve having a reflectance of at least 60% for each wavelength from about 440 nm to about 580 nm.
Aspect 76 provides an optical filter stack of any one of Aspects 1-75, which is configured for a reflectance curve having a reflectance of at least 60% for each wavelength from about 450 nm to about 580 nm.
Aspect 76 provides an optical filter stack of any one of Aspects 1-75, which is configured for a reflectance curve having a reflectance of at least 70% for each wavelength from about 460 nm to about 570 nm.
Aspect 76 provides an optical filter stack of any one of Aspects 1-75, which is configured for a reflectance curve having a reflectance of at least 70% for each wavelength from about 500 nm to about 570 nm.
Aspect 76 provides an optical filter stack of any one of Aspects 1-75, which is configured for a reflectance curve having a reflectance of at least 80% for each wavelength from about 540 nm to about 560 nm.
Aspect 76 provides an optical filter stack, which comprises layers that comprise or consist of hafnium, silicon, scandium, aluminum, or any combination thereof.
Aspect 76 provides an apparatus for inactivating a pathogen in air, comprising a far-UV radiation source that emits radiation through a window comprising the optical filter stack of any one of Aspects 1-75.
Aspect 77 provides an apparatus for inactivating a pathogen in air, comprising a far-UV radiation source that emits radiation through a window comprising an optical filter stack having plurality of plurality of layers of a high refractive index dielectric material and layers a low refractive index dielectric material each having an optical thickness configured to together provide an emission curve with a peak emission at about 222 nm, and an emission at one or more wavelengths from 250 nm to 265 nm having an intensity of 0.1% or more of the emission at 222 nm, as measured upon treatment with an unfiltered KrCl excimer emission, as measured at an incident angle of zero degrees.
Aspect 78 provides an apparatus of Aspects 76 or 77, wherein the emission curve has one or more wavelengths from 237 nm to 245 nm having an intensity of 1% or more of the emission at 222 nm.
Aspect 7 provides an apparatus of any one of Aspects 76 to 78, wherein the emission curve has one or more wavelengths between 241 nm and 245 nm having an intensity of 1% or more of the emission at 222 nm, and an emission at 258 nm having an intensity of 0.1% or more of the emission at 222 nm, as measured upon treatment with an unfiltered KrCl excimer emission, as measured at an incident angle of zero degrees.
Aspect 80 provides an apparatus of any one of Aspects 76 to 79, wherein the emission curve has a peak emission at about 222 nm, an emission at one or more wavelengths from 237 nm to 245 nm having an intensity of 1% or more of the emission at 222 nm, and an emission at one or more wavelengths from 250 nm to 265 nm having an intensity of 0.1% or more of the emission at 222 nm, as measured upon treatment with an unfiltered KrCl excimer emission, as measured at an incident angle of 30 degrees.
Aspect 81 provides an apparatus of any one of Aspects 76 to 80, wherein the emission curve has an emission at one or more wavelengths between 241 nm and 245 nm having an intensity of 1% or more of the emission at 222 nm, and an emission at 258 nm having an intensity of 0.1% or more of the emission at 222 nm, as measured upon treatment with an unfiltered KrCl excimer emission, as measured at an incident angle of 30 degrees.
Aspect 82 provides an apparatus of any one of Aspects 76 to 81, wherein the window is configured to receive radiation at an incident angle of zero degrees to at least 30 degrees.
Aspect 83 provides an apparatus of any one of Aspects 76 to 82, wherein more than 80% of the radiation incident to the window has an angle of greater than 20 degrees.
Aspect 84 provides an apparatus of any one of Aspects 76 to 83, wherein the window is substantially planar.
Aspect 85 provides a method for inactivating a pathogen in air, comprising irradiating air in a human-use space with UV radiation having a peak emission at 222 nm, an emission at one or more wavelengths from 237 nm to 245 nm having an intensity of 1% or more of the emission at 222 nm, and an emission at one or more wavelengths from 250 nm to 265 nm having an intensity of 0.1% or more of the emission at 222 nm.
Aspect 86 provides a method of Aspect 85, wherein the UV radiation has an emission at 240 nm having an intensity of 1% or more of the emission at 222 nm.
Aspect 87 provides a method of Aspect 85 or Aspect 86, wherein the UV radiation has an emission at 241 nm having an intensity of 1% or more of the emission at 222 nm.
Aspect 88 provides a method of any one of Aspects 85 to 87, wherein the UV radiation has an emission at 242 nm having an intensity of 1% or more of the emission at 222 nm.
Aspect 89 provides a method of any one of Aspects 85 to 88, wherein the UV radiation has an emission at 243 nm having an intensity of 1% or more of the emission at 222 nm.
Aspect 90 provides a method of any one of Aspects 85 to 89, wherein the UV radiation has an emission at 258 nm having an intensity of 0.1% or more of the emission at 222 nm.
Aspect 90 provides an optical filter stack, an apparatus, or method incorporating any combination or permutation of one or more of the aforementioned features in Aspects 1-89.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed:

1. An optical filter stack having layers of a high index dielectric material and layers of a low index dielectric material, each layer having an optical thickness configured to together provide a transmittance curve having a transmittance of at least 70% at 222 nm and substantial transmittance of one or more wavelength from 240 nm to 280 nm, as measured at an incident angle of zero degrees.

2. The optical filter stack of claim 1, wherein the transmittance curve has one or more wavelength from 230 nm to 245 nm with a transmittance about the same or greater than the transmittance at 222 nm, and each wavelength from 255 nm to 265 nm has a transmittance of less than 15% of the transmittance at 222 nm, as measured at an incident angle of zero degrees.

3. The optical filter stack of claim 1, wherein the transmittance curve has a transmittance at one or more wavelengths from 240 nm to 245 nm that is about 85% or more of a peak transmittance from 222 nm to 245 nm, and a transmittance at one or more wavelengths from 255 nm to 265 nm that is about 15% or less of the peak transmittance from 222 nm to 245 nm, as measured at an incident angle of zero degree.

4. The optical filter stack of claim 1, wherein the transmittance curve has a transmittance at one or more wavelength from 242 nm to 252 that is 50% of a peak transmittance from 222 nm to 242 nm, and a transmittance at one or more wavelengths from 255 nm to 265 nm that is at least 3% of the peak transmittance from 222 nm to 245 nm, as measured at an incident angle of zero degrees.

5. The optical filter stack of claim 1, wherein the transmittance curve has a transmittance at one or more wavelengths from 255 nm to 265 nm that is 3% to 15% of a peak transmittance from 222 nm to 245 nm, and a transmittance at one or more wavelengths from 270 nm to 370 that is 90% or greater than the peak transmittance from 222 nm to 245 nm, as measured at an incident angle of zero degrees.

6. The optical filter stack of claim 1, wherein the transmittance curve has a transmittance at one or more wavelengths from 270 nm to 370 nm that is 90% or greater than the peak transmittance from 222 nm to 230 nm, as measured at an incident angle of zero degrees.

7. The optical filter stack of claim 1, wherein the transmittance curve has one or more wavelength from 230 nm to 370 nm with a transmittance about the same or greater than the transmittance at 222 nm, as measured at an incident angle of zero degrees.

8. The optical filter stack of claim 1, wherein the transmittance curve has one or more wavelength from 237 nm to 370 nm with a transmittance about the same or greater than the transmittance at 222 nm, as measured at an incident angle of zero degrees.

9. The optical filter stack of claim 1, wherein each wavelength from 230 nm to 241 nm of the transmittance curve has a transmittance at least 60%, as measured at an incident angle of zero degrees.

10. The optical filter stack of claim 1, wherein each wavelength from 230 nm to 242 nm of the transmittance curve has a transmittance at least 50%, as measured at an incident angle of zero degrees.

11. The optical filter stack of claim 1, wherein each wavelength from 230 nm to 245 nm of the transmittance curve has a transmittance at least 25% of the transmittance at 222 nm, as measured at an incident angle of zero degrees.

12. The optical filter stack of claim 1, wherein each wavelength from 230 nm to 248 nm of the transmittance curve has a transmittance at least 10% of the transmittance at 222 nm, as measured at an incident angle of zero degrees.

13. The optical filter stack of claim 1, wherein each wavelength from 230 nm to 250 nm of the transmittance curve has a transmittance at least 5%, as measured at an incident angle of zero degrees.

14. The optical filter stack of claim 1, wherein each wavelength from 230 nm to 260 nm of the transmittance curve has a transmittance at least 3% of the transmittance at 222 nm, as measured at an incident angle of zero degrees.

15. The optical filter stack of claim 1, which has about 7 to about 30 layers.

16. The optical filter stack of claim 1, which has 16 or fewer layers.

17. The optical filter stack of claim 1, wherein the low index dielectric material is SiO₂ and the high index dielectric material is HfO₂.

18. The optical filter stack of claim 1, which further comprises additional reflective layers between the low index dielectric material and the high index dielectric material, and wherein the optical filter stack is configured to reflect visible light.

19. An apparatus for inactivating a pathogen in air, comprising a far-UV radiation source that emits radiation through a window comprising the optical filter stack of claim 1.

20. An apparatus for inactivating a pathogen in air, comprising a far-UV radiation source that emits radiation through a window comprising an optical filter stack having layers of a high index dielectric material and layers of a low index dielectric material each having an optical thickness configured to together provide a peak emission at about 222 nm, and an emission at one or more wavelengths from 250 nm to 265 nm having an intensity of 0.1% or more of the emission at 222 nm, as measured upon treatment with an unfiltered KrCl excimer emission, as measured at an incident angle of zero degrees.
